(19) United States
(12) Patent Application Publication
(10) Pub. No.: US 2004/0138436 A1
(43) Pub. Date: Jul. 15, 2004

(54) Water-soluble salt of sulfonamides as colorants for the pigmenting of porous materials and for use in inkjet printing

(76) Inventors: Hans-Thomas Schacht, Rheinfelden (DE); Gilbert Moegle, Magstatt-le-Bas (FR)

Correspondence Address: CIBA SPECIALTY CHEMICALS CORPORATION, PATENT DEPARTMENT, 540 WHITE PLAINS RD, P O BOX 2005, TARRYTOWN, NY, 10591-9005, US

(21) Appl. No.: 10/474,937

(22) PCT Filed: Apr. 11, 2002
(86) PCT No.: PCT/EP02/04071

(30) Foreign Application Priority Data
Apr. 19, 2001 (EP) 01810383.8

(51) Int. Cl. C07D 221/22; C 07D 4 3/02; C09B 29/00; C09B 7/02

(52) U.S. Cl. 534/844; 544/76; 546/37; 546/49; 548/457; 540/133; 548/453; 552/225

(57) ABSTRACT

The present invention relates to colorant of the general formula, wherein A, B and B′, m and m′ and n and n′ are as defined in the description. The colorants can be used for the pigmenting of porous materials and in injekt printing and, in the case of pigmenting, are converted to the insoluble pigment form. In the pigmenting of porous materials, for example in the pigmenting of wood or anodised aluminium in the pores, the colorants yield very good penetration and fastness to light and, in injekt printing, yield very good water-resistance.

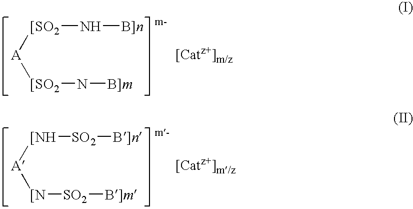

The present invention relates to water-soluble salts of sulfonamides that can be used as colorants for the pigmenting of porous materials and in inkjet printing and, in the case of pigmenting, are converted to the insoluble pigment form. In the pigmenting of porous materials, for example in the pigmenting of wood or anodised aluminium in the pores, the water-soluble salts of sulfonamides yield very good penetration and fastness to light and, in inkjet printing, yield very good water-resistance.

DE-A-2757226 describes a method of dyeing fibre materials consisting wholly or partially of cellulose, wherein a mono- or dis-azo dye having a sulfonamide group is applied to the fibre material in an alkaline aqueous solution and is fixed to the fibre material by means of subsequent acid treatment in the acid pH range. The dyeings exhibit good in-use fastness properties, especially fastness to washing.

Water-soluble phthalocyanine dyes are described in EP-A-0 024 677. They are substituted by at least one —SO₂—NH—CN group and may contain an —SO₂—NY¹Y² group wherein Y¹ and Y² denote a hydrogen atom or an unsubstituted or substituted alkyl group. The phthalocyanine dyes are suitable as water-soluble dyes for the dyeing and printing of fibre materials, it being preferred to use them in the form of their ammonium salts. They are generally applied in a neutral or acidic aqueous medium and are fixed to the material by means of moderate or high temperature.

U.S. Pat. No. 3,972,904 relates to water-soluble sodium or ammonium salts of the following formula

F-A-C(O)—N(Me)SO₃Me,

wherein F is the radical of a water-insoluble dye, A is —O—, —S—, —NY³—(Y³═H, alkyl, etc.) and Me is an alkali metal atom or an ammonium ion. The water-insoluble dyes must contain an OH—, SH— or NHY³ group. The salts are used for dyeing polyester. The regeneration of the water-soluble dye is achieved by heating.

DE-A-197 11 445 relates to dye salts of formula

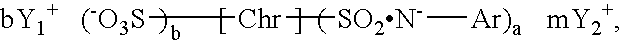

wherein Chr is an (m+n)-valent radical of a chromophore from the series of phthalocyanines that are metal-free or contain metal, quinacridones, mono-, dis- or poly-azo dyes, anthraquinones or copper formazanes, Ar is unsubstituted or substituted phenyl or unsubstituted or substituted naphthyl, Y₁⁺ is a metal cation or ammonium ion, Y₂⁺ is a proton, a metal cation or ammonium ion, a is from 1 to 6 and b is from 1 to 6, the sum of a and b being a maximum of 6, and to the use of the dye salts in the dyeing of polymeric material.

DE-A-199 58 181 discloses diketopyrrolopyrrole-based pigment dispersants that contain sulfonamide groups in addition to sulfonic acid groups.

GB-1 198 501 relates to a method of dyeing polyester using dyes of the formula phthalocyanine-(Y-Y⁴)ₙ wherein Y can be an —SO₂NH—, CH₂SO₂—NH-phenylene-O— or CH₂SO₂NH-phenylene-C(O)O— group and Y⁴ denotes a lower alkyl or acyloxy-lower alkyl group (see Examples 10, 11 and 21).

U.S. Pat. No. 4,000,965 discloses that stable solutions of 1:2 chromium or 1:2 cobalt complexes of monoazo, disazo or polyazo dyes or azomethine dyes can be obtained in organic solvents that contain hydroxy groups, or in mixtures thereof with water in the presence of alkaline lithium salts. There are described sodium salts of metal-free intermediates of metal complex dyes that carry an —SO₂NY⁵ Na group, wherein Y⁵ can be a hydrogen atom or an alkyl, aryl or acyl radical, and that are converted to the metal complex dyes in accordance with the following equation:

8{H₂F¹—SO₂NY⁵}⁻Na⁺+O₂+4LiOH+4CoCl₂→

{4F¹—NH—SO₂—Y⁵═Co═F¹—SO₂NHY⁵}⁻Li⁺+8NaCl+6H₂O

wherein Y⁵ is as defined above and F¹ is the radical of one of the above-mentioned dyes (see also U.S. Pat. No. 3,617,176).

DE-A-2545393 describes dye salts, free of complex-bound metal, having an SO₃Met group or an SO₂—NMet-SO₂ group, wherein Met is an alkali metal, and at least one further acid group, at least 20% of which has been converted to the alkali salt, which acid group forms alkali salts only at a pH greater than 8.

Suitable acid groups that form alkali salts only at a pH>8 are phenolic and enolic OH groups and, advantageously, sulfonic acid amide groups of formula —SO₂NH—Y⁶, wherein Y⁶ is hydrogen, unsubstituted or substituted C₁₋₄alkyl, aryl or aralkyl. The dye salts are very readily soluble in both hot water and cold water.

Numerous pigments modified with sulfonamide groups have been described. The conversion of the sulfonamide groups to the salt form and the use of pigments modified in such a manner for the pigmenting of porous materials and in inkjet printing, however, have not yet been described.

WO-98/58027 (porous materials in general), WO00/36210 (wood), EP00/09376 (wood), EP-A-1044945 (pencil leads), WO00/17275 (colour filters) and WO0/27930 (metal oxides) relate to the pigmenting of porous materials, starting from soluble pigment precursors.

The problem of the present invention is accordingly to provide water-soluble colorants that can be converted readily to the insoluble pigment form and, in the pigmenting of porous materials, for example wood, yield very good penetration and very good fastness to light and, in inkjet printing, yield very good water-resistance.

Surprisingly, it has now been found that the problem is solved by using salts of primary or secondary sulfonamide groups as solubilising groups.

The present invention accordingly relates to colorants of the general formula

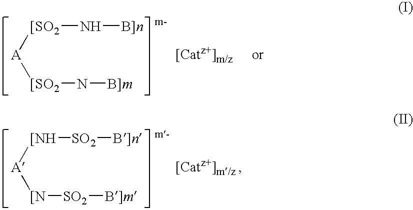

wherein n and n′ denote a value from 0 to 4,

m and m′ denote a value from 1 to 8, the sum of m+n and of m′ and n′ being less than or equal to 8,

z is an integer from 1 to 5, especially 1,

A is the radical of a chromophore of the series 1-aminoanthraquinone, anthraquinone, anthrapyrimidine, azo, azomethine, benzodifuranone, quinacridone, quinacridonequinone, quinophthalone, diketopyrrolopyrrole, dioxazine, flavanthrone, indanthrone, indigo, isoindoline, isoindolinone, isoviolanthrone, perinone, perylene, phthalocyanine, pyranthrone or thioindigo,

A′ is the radical of a chromophore that already contains one or more primary amino groups, such as 1-aminoanthraquinone, or A′ is one of the chromophore radicals listed under A modified with from 1 to 8, preferably with from 1 to 4, amino groups,

Cat is an alkali metal cation or an ammonium cation and

B and B′ are each independently of the other a branched or straight-chain C₁₋₈alkyl, C₂₋₈alkenyl, C₂₋₈alkynyl, aryl, N-, O- or S-containing 5- or 6-membered heterocyclic ring, C₁₋₈alkylarylene, aryl-C₁₋₈alkylene or aryl-L-arylene radical, which may be substituted by one or more groups —OH, —OCat, —SH, —SCat, —OR¹, —SR², —C(O)OR³, —C(O)R⁴ and/or —NR⁵R⁶, it being possible for the C₁₋₈alkyl radical to be uninterrupted or interrupted one or more times by —O— or by —S—,

R¹, R², R³ and R⁴ are each independently of the others a C₁₋₈alkyl radical, a C₇₋₁₁aralkyl radical or a C₆₋₁₀aryl radical and R⁴ can additionally be a hydrogen atom,

L is a bond, —NR⁷, wherein R⁷ is a hydrogen atom or a C₁₋₁₄alkyl radical, or an —N═N— group, and R⁵ and R⁶ are each independently of the other a hydrogen atom, a C₁₋₈alkyl radical, a C₁₋₄alkoxy-C₁₋₄alkyl radical, a C₆₋₁₀aryl radical, a C₇₋₁₁ aralkyl radical or a radical —(CH₂)ₒOH, wherein o is an integer from 2 to 6, and B can additionally be a hydrogen atom, it being possible for B and B′ within a chromophore A or A′ to have different substituent meanings.

The present invention relates also to the use of colorants of the general formulae I and II for the pigmenting of porous materials, especially for the pigmenting of wood or of anodised aluminium in the pores, and, in inkjet printing, to a method of pigmenting a porous material, especially for the pigmenting of wood or anodised aluminium in the pores, comprising

a) treatment of the substrate with an aqueous solution of the colorant of general formula I or II and

b) conversion of the colorant to a pigment of formula I′ or II′, respectively,

A-[SO₂—NH—B]n+m (I′) or

A′-[NH—SO₂—B′]n′+m′ (II′),

wherein A and A′, B and B′, m and m′ and n and n′ are as defined above, and to porous materials that are obtainable by that method, and to porous materials that include pigments of general formula I′ or II′.

Insoluble pigments that have a secondary or primary sulfonamide group can be rendered water-soluble in the form of a salt, especially in the form of the ammonium, sodium or potassium salt. The colorant is converted back to the water-insoluble pigment form by protonation, for example by the addition of acids.

Ligneous materials that have been pigmented using the colorants according to the invention, even in highly dilute concentrations, exhibit very good penetration and very good fastness to light. In the pigmenting of anodised aluminium good results are also obtained with the colorants according to the invention, that is to say especially homogeneous coloration and very good fastness to light. In inkjet printing, special mention may be made in particular of the very good water-resistance of the colorants according to the invention.

In groups B and B′, the radicals can have the following meanings: alkyl or alkylene can be straight-chained or branched.

C₁₋₈alkyl is, for example, methyl, ethyl, n-propyl, isopropyl, n-butyl, sec-butyl, isobutyl, tert-butyl, n-pentyl, 2-pentyl, 3-pentyl, 2,2-dimethylpropyl, hexyl, heptyl, 2,4,4-trimethylpentyl, 2-ethylhexyl or octyl, which may be unsubstituted or substituted by —OH, —OCat, —SH, —SCat, —OR¹, —SR², —C(O)OR³, —C(O)R⁴ or by —NR⁵R⁶, wherein R¹, R², R³, R⁴, R⁵ and R⁶ are as defined above.

Examples of C₂₋₈alkenyl, which may also contain two double bonds which may be isolated or conjugated, are vinyl, allyl, 2-propen-2-yl, 2-buten-1-yl, 3-buten-1-yl, 1,3-butadien-2-yl, 2-penten-1-yl, 3-penten-2-yl, 2-methyl-1-buten-3-yl, 2-methyl-3-buten-2-yl, 3-methyl-2-buten-1-yl or 1,4-pentadien-3-yl, which may be unsubstituted or substituted by —OH, —OCat, —SH, —SCat, —OR¹, —SR², —C(O)OR³, —C(O)R⁴ or by —NR⁵R⁶, wherein R¹, R², R³, R⁴, R⁵ and R⁶ are as defined above.

Preference is given especially to linear C₁₋₅alkyl and C₂₋₅alkenyl radicals terminally substituted by a group —OH, —OCat, —SH, —SCat, —OR¹, —SR², —C(O)OR³, —C(O)R⁴ or —NR⁵R⁶. C₂-C₈Alkyl that is interrupted one or more times by —O— or by —S— is, for example, interrupted 1, 2 or 3 times by —O— and/or by —S—, resulting, for example, in structural units such as —(CH₂)₂OCH₃, —(CH₂CH₂O)₂CH₂CH₃, —CH₂—O—CH₃, —CH₂CH₂—O—CH₂CH₃, —[CH₂CH₂O]_{y}—CH₃, wherein y=1-3, —CH₂—CH(CH₃)—O—CH₂—CH₂CH₃ or —CH₂—CH(CH₃)—O—CH₂—CH₃, which may be unsubstituted or substituted by —OH, —OCat, —SH, —SCat, —OR¹, —SR², —C(O)OR³, —C(O)R⁴ or by —NR⁵R⁶.

Examples of C₂₋₈alkynyl are ethynyl, 1-propyn-1-yl, 2-butyn-1-yl, 3-butyn-1-yl, 2-pentyn-1-yl and 3-pentyn-2-yl.

C₁-C₈Alkylene is linear or branched alkylene, such as, for example, methylene, ethylene, propylene, isopropylene, n-butylene, sec-butylene, isobutylene, tert-butylene, pentylene, hexylene, heptylene, —CH(CH₃)—CH₂—, —CH(CH₃)—(CH₂)₂—, —CH(CH₃)—(CH₂)₃—,

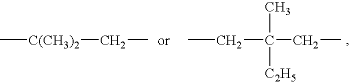

wherein alkylene radicals having from 1 to 5 carbon atoms are preferred.

An alkylene radical interrupted by —O— or by —S— results, for example, in structural units such as —CH₂—O—CH₂—, —CH₂CH₂—O—CH₂CH₂—, —CH₂—CH(CH₃)—O—CH₂—CH(CH₃)—, —CH₂—S—CH₂—, —CH₂CH₂—S—CH₂CH₂— or —CH₂CH₂CH₂—S—CH₂CH₂CH₂—.

Examples of a C₁₋₈alkoxy radical, which may be linear or branched, are methoxy, ethoxy, n-propoxy, isopropoxy, n-butoxy, sec-butoxy, isobutoxy, tert-butoxy, n-pentyloxy, 2-pentyloxy, 3-pentyloxy, 2,2-dimethylpropoxy, n-hexyloxy, n-heptyloxy, n-octyloxy, 1,1,3,3-tetramethylbutoxy and 2-ethylhexyloxy. According to the present invention, aryl is understood to mean especially an aryl radical having from 6 to 12 carbon atoms, examples thereof being phenyl, naphthyl and biphenyl, which may be substituted one, two or three times by linear or branched C₁₋₄alkyl, such as methyl, ethyl, n-propyl, isopropyl, n-butyl, isobutyl, sec-butyl or tert-butyl, by linear or branched C₁₋₄alkoxy, such as methoxy, ethoxy, n-propoxy, isopropoxy, n-butoxy, isobutoxy, sec-butoxy or tert-butoxy, by linear or branched C₁₋₄alkylthio, such as methylthio, ethylthio, n-propylthio, isopropylthio, n-butylthio, isobutylthio, sec-butylthio or tert-butylthio, —OH, —SH, —OCat,

—SCat or by a group (CH₂)e-E, wherein e is an integer from 1 to 6, especially 2 or 3, and

E is a hydrogen atom, a group —OH, —OCat, —SH, —SCat, —OR¹, —SR², —C(O)OR³, —C(O)R⁴ or —NR⁵R⁶, wherein R¹, R², R³ and R⁴ are each independently of the others a C₁₋₄alkyl radical, especially methyl or ethyl, and R⁵ and R⁶ are a radical —(CH₂)ₒOH, wherein o is an integer from 2 to 6, especially 2 or 3, and Cat is an alkali metal cation, especially a sodium or potassium cation, unsubstituted ammonium or an ammonium cation.

Preference is given to phenyl groups which may be substituted by one, two or three groups selected from —OH, methoxy, (CH₂)₂OH, —OCat and (CH₂)₂OCat, such as, for example, 3,4,5-trimethoxyphenyl, 4-hydroxyphenyl, 3-hydroxy-4-methoxyphenyl and 2-hydroxy-1-ethylphenyl.

Examples of a C₇₋₁₁aralkyl radical which may be unsubstituted or substituted are benzyl, 2-benzyl-2-propyl, β-phenyl-ethyl, α,α-dimethylbenzyl and ω-phenyl-butyl.

An O-, S- or N-containing 5- or 6-membered heterocyclic ring is, for example, pyrrolyl, oxinyl, dioxinyl, 2-thienyl, 2-furyl, 1-pyrazolyl, 2-pyridyl, 2-thiazolyl, 2-oxazolyl, 2-imidazolyl, isothiazolyl, triazolyl or any other ring system consisting of thiophene, furan, pyridine, thiazole, oxazole, imidazole, isothiazole, thiadiazole, triazole, pyridine and benzene rings that is unsubstituted or substituted by from 1 to 6 ethyl, methyl, ethylene and/or methylene substituents.

B and B′ within a chromophore A or A′ can have different substituent meanings, that is to say, according to the invention, for example, a copper phthalocyanine of the following formula is also included:

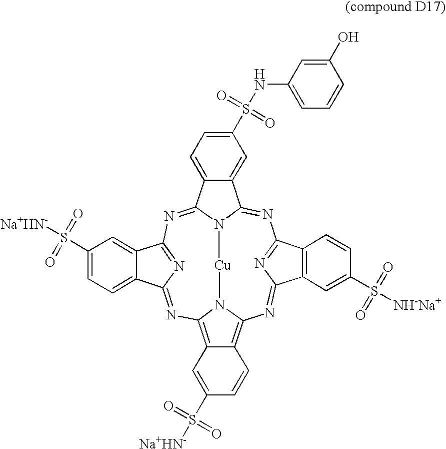

Suitable as anion Cat in the formulae (I) and (II)—and also in the groups —OCat and —SCat—are generally radicals that can form water-soluble salts with the sulfonamides. These are, for example, alkaline earth metal cations, such as strontium or calcium cations, alkali metal cations, especially lithium, sodium and potassium cations, and quaternary ammonium cations, especially unsubstituted ammonium and ammonium cations of the formula ⁺NR³¹R³²R³³R³⁴, wherein R³¹, R³², R³³ and R³⁴ are each independently of the others a hydrogen atom, a straight-chain or branched C₁₋₁₆alkyl radical, which may be unsubstituted or substituted by one or more C₁₋₄alkoxy radicals, a straight-chain or branched C₂₋₁₆alkenyl radical, a hydroxy-C₁₋₈alkyl radical, especially a hydroxy-C₁₋₄alkyl radical, or a C₆₋₁₂aryl radical unsubstituted or substituted by one or more C₁₋₄alkyl radicals, C₁₋₄alkoxy radicals or hydroxy groups, especially a phenyl group substituted by a hydroxy group, or a C₇₋₁₁aralkyl radical, such as phenyl-C₁₋₄alkyl, wherein at least one of the radicals R³¹, R³², R³³ and R³⁴ is other than a hydrogen atom, or two of the radicals R³¹, R³², R³³ and R³⁴ together with the nitrogen atom to which they are bonded form a 5- or 6-membered ring, which may contain additional hetero atoms, such as, for example, S, N or O.

Examples of especially preferred ammonium cations are:

unsubstituted ammonium,

mono-, di-, tri- or tetra-C₁₋₄alkylammonium, such as methylammonium, ethylammonium, 3-propylammonium, isopropylammonium, butylammonium, sec-butylammonium, isobutylammonium, 1,2-dimethylpropylammonium or 2-ethylhexylammonium, dimethylammonium, diethylammonium, dipropylammonium, diisopropylammonium, dibutylammonium, diisobutylammonium, di-sec-butylammonium, di-2-ethylhexyl-ammonium, N-methyl-n-butylammonium or N-ethyl-n-butylammonium, trimethyl- or triethyl-ammonium, tripropylammonium, tributylammonium, N,N-dimethylethyl-ammonium, N,N-dimethylisopropylammonium, N,N-dimethylbenzylammonium or (CH₃)₂((CH₃O)₂CHCH₂)NH⁺,

mono-, di-, tri- or tetra-C₈₋₁₆alkylammonium, such as

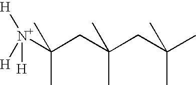

(idealised representation of the ammonium cation of Primene 81R®),

C₁₋₄alkoxy-C₁₋₄alkylammonium, such as 2-methoxyethylammonium, bis(2-methoxyethyl)ammonium, 3-methoxypropylammonium or ethoxypropylammonium,

mono-, di- or tri-(hydroxy-C₁₋₄alkyl)ammonium, such as mono-, di- or tri-ethanolammonium, mono-, di- or tri-isopropanolammonium, N-methyl- or N,N-dimethylethanolammonium, -propanolammonium or -isopropanolammonium, N-methyldiethanolammonium, -dipropanolammonium or -diisopropylammonium, N-ethyldiethanolammonium, -dipropanolammonium or -diisopropylammonium, N-propyldiethanolammonium, -dipropanolammonium or -diisopropylammonium,

N-(2-hydroxyethyl)pyrrolidinium, N-(2- or 3-hydroxypropyl)pyrrolidinium, N-(2-hydroxyethyl)piperidinium, N-(2- or 3-hydroxypropyl)piperidinium, N-(2-hydroxyethyl)morpholinium, N-(2- or 3-hydroxypropyl)morpholinium or N-(2-hydroxyethyl)piperazinium, and

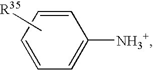

especially

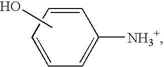

such as 2-, 3- or 4-hydroxyphenylammonium, wherein R³⁵ is a hydroxy group, a C₁₋₈alkoxy group, a carboxylic acid group or COOR³⁶, wherein R³⁶ is a C₁₋₈alkyl group, a C₆₋₁₀aryl group or a C₇₋₁₁aralkyl group. Tetramethylammonium and tetraethylammonium salts are especially suitable for inkjet printing. Ammonium cations of formula

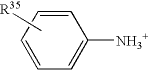

can contribute to an increase in fastness to light.

Also suitable are polyammonium salts, especially diammonium compounds. Preferred diammonium compounds are derived from the following amines: 1,2-diaminoethane, 1,2-diamino-1-methylethane, 1,2-diamino-1,2-dimethylethane, 1,2-diamino-1,1-dimethylethane, 1,2-diaminopropane, 1,3-diaminopropane, 1,3-diamino-2-hydroxypropane, N-methyl-1,2-diaminoethane, 1,4-diazacyclohexane, 1,2-diamino-1,1-dimethylethane, 2,3-diaminobutane, 1,4-diaminobutane, N-hydroxyethyl-1,2-diaminoethane, 1-ethyl-1,3-diaminopropane, 2,2-dimethyl-1,3-diaminopropane, 1,5-diaminopentane, 2-methyl-1,5-diaminopentane, 2,3-diamino-2,3-dimethylbutane, N-2-aminoethylmorpholine, 1,6-diaminohexane, 1,6-diamino-2,2,4-trimethylhexane, N,N-dihydroxyethyl-1,2-diaminoethane, N,N-dimethyl-1,2-diaminoethane, 4,9-dioxa-1,12-diaminododecane, 1,2-diaminocyclohexane, 1,3-diamino-4-methylcyclohexane, 1,2-diaminocyclohexane, 1-amino-2-aminomethyl-2-methyl-4,4-dimethylcyclohexane, 1,3-diaminomethylcyclohexane, N-2-aminoethylpiperazine, 1,1-di(4-aminocyclohexyl)methane, 1,1-di(4-aminophenyl)methane, N,N′-diisopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methyl-heptyl)-p-phenylenediamine, N,N′-dicyclohexyl-p-phenylenediamine, N,N′-diphenyl-p-phenylenediamine, N,N′-di(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine and N,N′-dimethyl-N,N′-di-sec-butyl-p-phenylenediamine.

In the groups —OR¹—SR², —C(O)OR³ and —C(O)R⁴, R¹, R², R³ and R⁴ can have, inter alia, the following meanings:

R¹, R², R³ and R⁴ as C₁₋₄alkyl are, for example, methyl, ethyl, n-propyl, isopropyl, n-butyl, isobutyl, sec-butyl or tert-butyl, preferably methyl or ethyl, and as C₁₋₁₂aryl are phenyl, biphenyl or naphthyl, preferably phenyl.

In the group —NR⁵R⁶, R⁵ and R⁶ are, in addition to a hydrogen atom, a C₁₋₄alkyl radical, e.g. methyl, ethyl, n-propyl, isopropyl, n-butyl, isobutyl, sec-butyl or tert-butyl, preferably methyl or ethyl, or a radical —(CH₂)ₒOH, wherein o is an integer from 1 to 6, especially 2 or 3, and the nitrogen atom is preferably symmetrically substituted.

B and B′ are especially preferably selected from the following groups:

a hydrogen atom

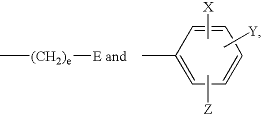

wherein e is an integer from 1 to 6, especially 2 or 3, E is a hydrogen atom, a group —OH, —OCat, —SH, —SCat, —OR¹, —SR², —NR⁵R⁶ or —C(O)OR³, and X, Y and Z are each independently of the others selected from a hydrogen atom and a group —OH, —OCat, —SH, —SCat, —OR¹, —SR², —NR⁵R⁵ and —C(O)OR³, wherein R¹, R² and R³ are each independently of the others a C₁₋₄alkyl radical, especially methyl or ethyl, and R⁵ and R⁶ denote a radical —(CH₂)ₒOH, wherein o is an integer from 2 to 6, and Cat is a sodium or potassium cation, unsubstituted ammonium or an ammonium cation described hereinbefore as preferred.

A′ is the radical of a chromophore that already contains one or more primary amino groups, such as 1-aminoanthraquinone, or is one of the chromophore radicals listed under A substituted by from 1 to 8, preferably by from 1 to 4, amino groups, such as, for example,

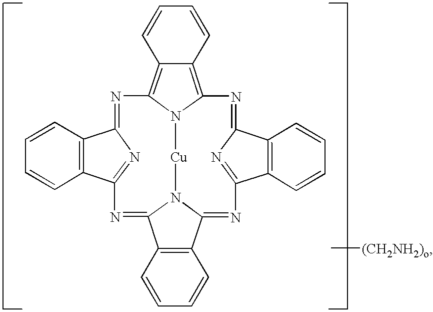

wherein o denotes a value from 1 to 8, preferably from 1 to 4 (see EP-A-311 562).

A is the radical of a known chromophore having the basic structure A(H)ₘ₊ₙ. Examples of such chromophores are described, for example, in W. Herbst, K. Hunger, Industrielle Organische Pigmente, 2nd completely revised edition, VCH 1995. In principle, all chromophores whose basic structure can be modified with one or more sulfonamide groups are suitable. The chromophore is usually selected from the series 1-aminoanthraquinone, anthraquinone, anthrapyrimidine, azo, azomethine, benzodifuranone, quinacridone, quinacridonequinone, quinophthalone, diketopyrrolopyrrole, dioxazine, flavanthrone, indanthrone, indigo, isoindoline, isoindolinone, isoviolanthrone, perinone, perylene, phthalocyanine, pyranthrone or thioindigo.

Examples of pigments (and substituted derivatives thereof) that can be used as starting compound for the sulfonamide salts according to the invention and that come under the classes of pigments mentioned above are described in W. Herbst, K. Hunger, Industrielle Organische Pigmente, 2nd completely revised edition, VCH 1995: 1-aminoanthraquinone pigments: p. 503-511; anthraquinone pigments: p. 504-506, 513-521 and 521-530; anthrapyrimidine: p. 513-415; azo pigments: p. 219-324 and 380-398; azomethine pigments: p. 402-411; quinacridone pigments: p. 462-481; quinacridonequinone pigments: p. 467-468; quinophthalone pigments: p. 567-570; diketopyrrolopyrrole pigments: p. 570-574; dioxazine pigments: p. 531-538; flavanthrone pigments: p. 517-519, 521; indanthrone pigments: p. 515-517; isoindoline pigments: p. 413-429; isoindolinone pigments: p. 413-429; isoviolanthrone pigments: p. 528-530; perinone pigments: p. 482-492; perylene pigments: p. 482-496; phthalocyanine pigments: p. 431-460; pyranthrone pigments: p. 522-526; thioindigo pigments (indigo pigments): p. 497-500, it also being possible to use mixtures of such pigments, including solid solutions.

Depending on the intended use, generally only some of the sulfonamide groups may be converted to the salt form. In the pigmenting of wood, it is preferred according to the invention to convert practically all of the sulfonamide groups present in the molecule into the salt form, that is to say in formulae I and II n and n′ are 0, whereas in the pigmenting of anodised aluminium it may be advantageous, for the purpose of obtaining a suitable pH value, to convert only some of the sulfonamide groups into the salt form, that is to say in formulae I and II n and n′ are ≧1.

Colorants of formula I are preferred to those of formula II.

Of the colorants of formula I, preference is given to the following, wherein B is as defined above and Cat is an alkali metal cation, especially a sodium or potassium cation, unsubstituted ammonium or an ammonium cation described hereinbefore as preferred:

salts of 1-aminoanthraquinone and anthraquinone pigments of formula

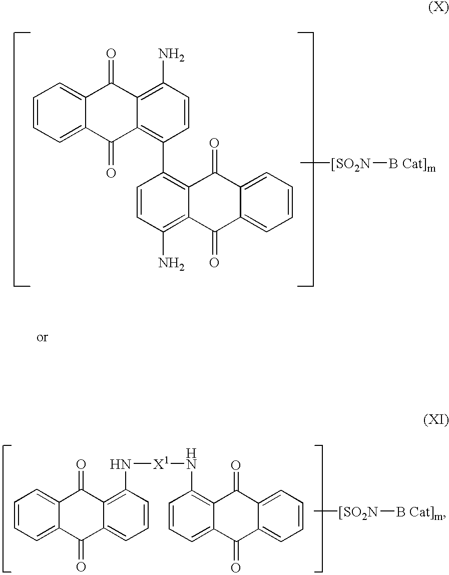

wherein X¹ is a group

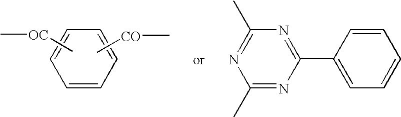

and m denotes a value from 1 to 4, especially from 2 to 3;

salts of quinacridone pigments of formula

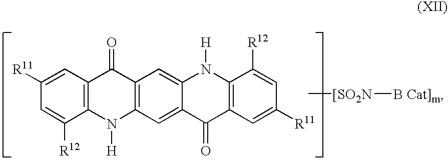

wherein R¹¹ and R¹² are each independently of the other hydrogen, halogen, C₁-C₂₄alkyl, C₁-C₆alkoxy or phenyl and m denotes a value from 1 to 4, especially from 2 to 3;

salts of pyrrolo[3,4-c]pyrroles of formula

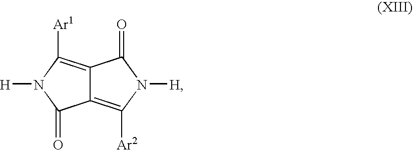

wherein Ar¹ and Ar² are each independently of the other a group of formula

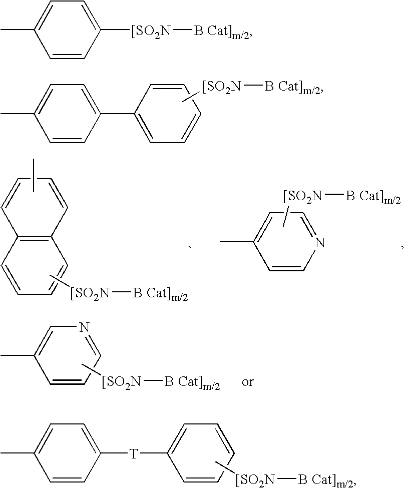

wherein T is —CH₂—, —CH(CH₃)—, —C(CH₃)₂—, —CH═N—, —N═N—, —O—, —S—, —SO—, —SO₂— or —NR¹³—, wherein R¹³ is hydrogen or C₁₋₆alkyl, especially methyl or ethyl, and m denotes a value from 1 to 4, especially from 2 to 3;

salts of dioxazines of formula

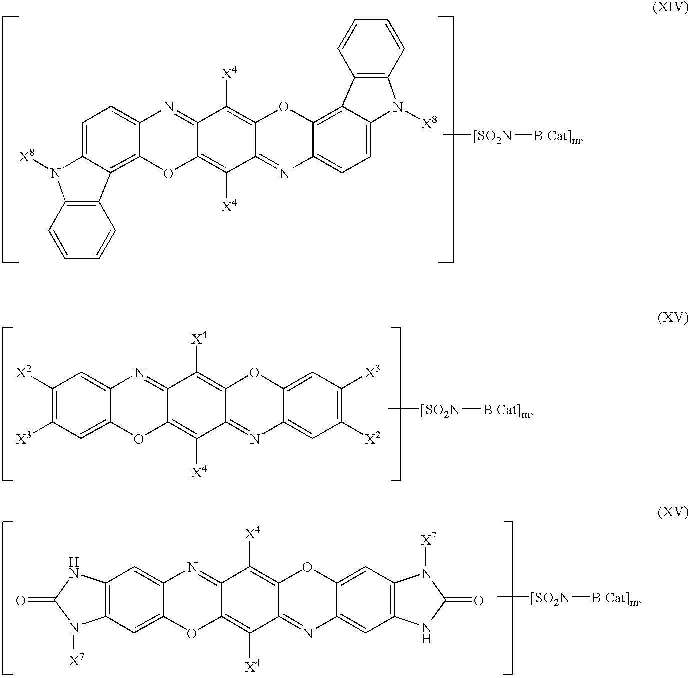

wherein X² is a C₁₋₄alkoxy radical, especially ethoxy, X³ is a C₁₋₄acylamino group, especially an acetylamino group, or a benzoylamino group and X⁴ is a chlorine atom or a radical NHC(O)CH₃, X⁷ is a hydrogen atom, a C₁₋₈alkyl radical, a substituted or unsubstituted phenyl, benzyl, benzanilide or naphthyl group, a C₅₋₇cycloalkyl radical or a radical of formula

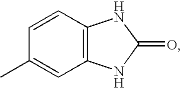

X⁸ is a hydrogen atom or a C₁₋₄alkyl radical and m denotes a value from 1 to 4;

salts of flavanthrones of formula

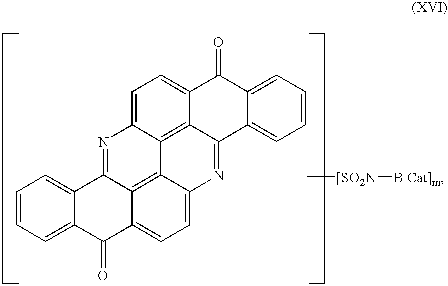

wherein m denotes a value from 1 to 4, especially from 2 to 3;

salts of indanthrones of formula

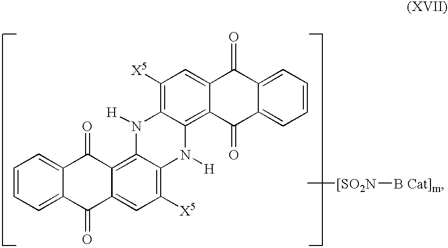

wherein X⁵ is a hydrogen or chlorine atom and m denotes a value from 1 to 4, preferably from 2 to 3;

salts of indigo derivatives of formula

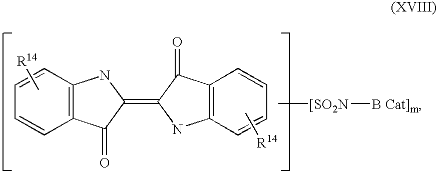

wherein R¹⁴ is hydrogen, CN, C₁₋₆alkyl, C₁₋₆alkoxy or halogen and m denotes a value from 1 to 3;

salts of isoviolanthrone:

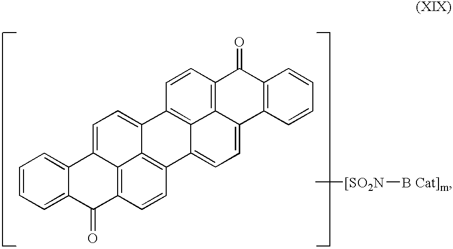

wherein m denotes a value from 1 to 4;

salts of perinone pigments of formula

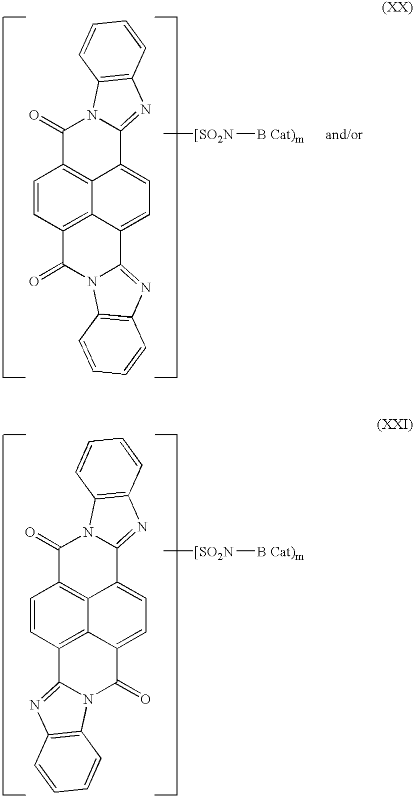

wherein m denotes a value from 1 to 4;

salts of perylene pigments of formula

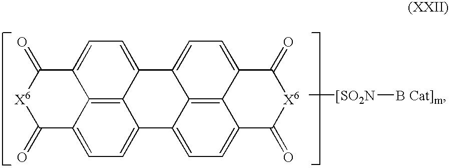

in which X⁶ is O or NR¹⁵, R¹⁵ being H, CH₃ or unsubstituted or substituted phenyl or C₇₋₁₁aralkyl, such as benzyl or 2-phenylethyl, and m denotes a value from 1 to 4, especially from 2 to 3, it being possible for the phenyl ring to be substituted by methyl, methoxy, ethoxy or by —N═N—Ph;

salts of phthalocyanines of formula

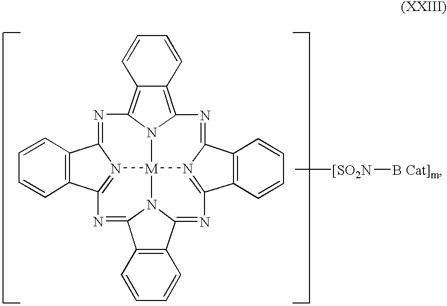

wherein M is H₂, a bivalent metal selected from the group Cu(II), Zn(II), Fe(II), Ni(II), Ru(II), Rh(II), Pd(II), Pt(II), Mn(II), Mg(II), Be(II), Ca(II), Ba(II), Cd(II), Hg(II), Sn(II), Co(II) and Pb(II), or a bivalent oxo metal selected from the group V(O), Mn(O) and TiO, and m denotes a value from 2 to 6, especially from 3 to 5;

salts of pyranthrone pigments of formula

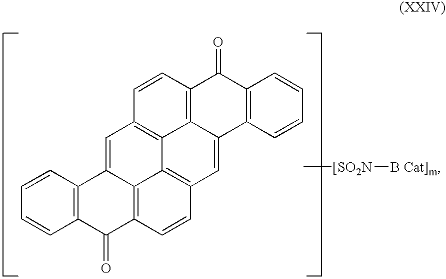

and bromo-, chloro- or bromo- and chloro-halogenated derivatives of the basic structure, for example the 2,10-dichloro, 4,6- and 6,14-dibromo derivatives, wherein m denotes a value from 2 to 4,

salts of thioindigo derivatives of formula

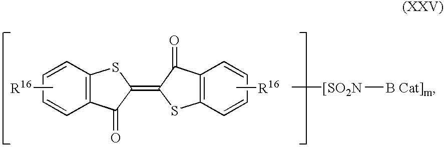

wherein R¹⁶ is hydrogen, CN, C₁₋₆alkyl, especially methyl, C₁₋₆alkoxy, especially methoxy, or halogen, especially chlorine, and m denotes a value from 1 to 3,

salts of monoazo yellow and orange pigments of formula

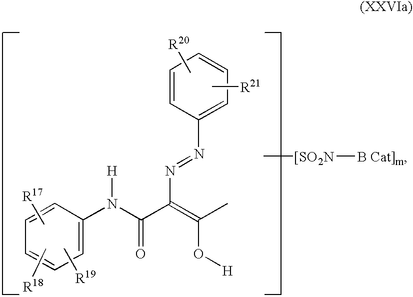

wherein m denotes a value from 1 to 4,

salts of diaryl yellow pigments of formula

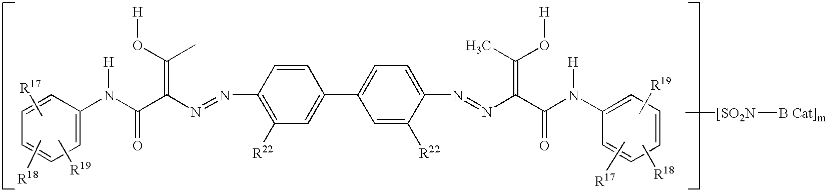

(XXVIb), wherein m denotes a value from 2 to 4,

salts of naphthol AS pigments of formula

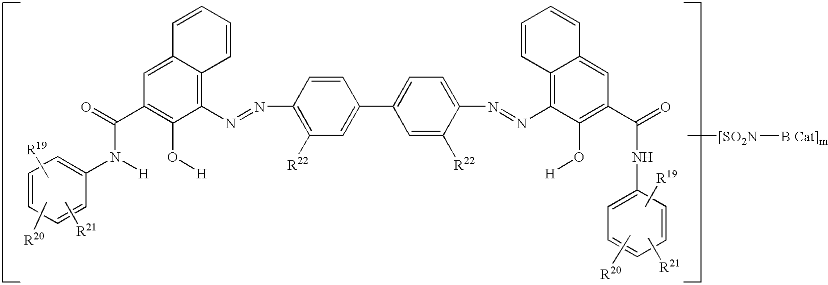

(XXVIc), wherein m denotes a value from 2 to 6,

R¹⁷ to R²¹ are each independently of the others a hydrogen atom, a halogen atom,

C₁₋₆-alkyl, C₁₋₆alkoxy, a nitro group or an acetyl group,

R²² is a hydrogen atom, a halogen atom, C₁₋₆alkyl or C₁₋₆alkoxy,

salts of mionoazoquinolone pigments of formula

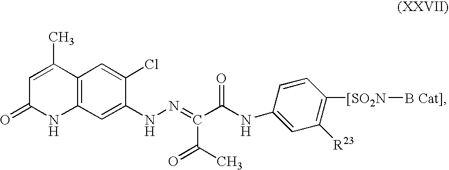

wherein R²³ is hydrogen, halogen, C₁₋₄alkyl, C₁₋₄alkoxycarbonyl, C₁₋₄alkylcarbonyl, C₁₋₄alkanoylamino (the preparation of monoazoquinolone pigments is described in EP01/12178), and

salts of azo pigments of formula

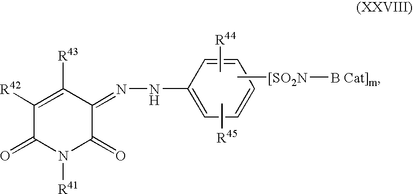

wherein

R⁴¹ is a hydrogen atom, a C₁₋₄alkyl radical, such as methyl or ethyl, or a perfluoro-C₁₋₄-alkyl radical, such as trifluoromethyl, a hydroxy-C₁₋₁₄alkyl radical or a C₁₋₈alkyl radical interrupted one or more times by —O—, such as CH₂CH₂CH₂—O—CH(CH₃)₂, a C₁₋₁₀aryl radical, such as phenyl, or a C₇₋₁₂aralkyl radical, such as benzyl,

R⁴² is a hydrogen atom, or a cyano or carbonamide group,

R⁴³ is a hydrogen atom, a carboxylic acid group or a salt thereof or a C₁₋₄alkyl radical,

R⁴⁴ and R⁴⁵ denote a C₁₋₄alkyl radical, such as methyl or ethyl, a perfluoro-C₁₋₄alkyl radical, such as trifluoromethyl, a C₁₋₄alkoxy radical, such as methoxy or ethoxy, a nitro group, a halogen atom, such as chlorine, COOR⁴⁶, wherein R⁴⁶ is a C₁₋₄alkyl radical, a C₆₋₁₀aryl radical that is unsubstituted or substituted, for example by one or two chlorine atoms, such as phenyl or 1,4-dichlorophenyl, or a C₇₋₁₂aralkyl radical, such as benzyl, CONHR⁴⁷, wherein R⁴⁷ is a C₁₋₄alkyl radical, a C₆₋₁₀aryl radical, such as phenyl, or a C₇₋₁₂aralkyl radical, such as benzyl, and m denotes a value from 1 to 2,

salts of isoindoline pigments of formula

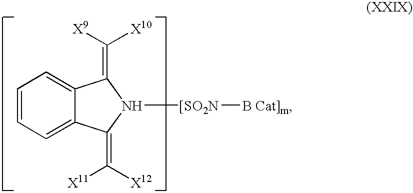

wherein

X⁹, X¹⁰, X¹¹ and X¹² are CN, CONH—C₁₋₈alkyl or CONH—C₆₋₁₀aryl or X⁹ and X¹⁰ and/or X¹¹ and X¹² are each members of a heterocyclic ring, such as

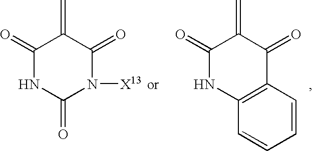

wherein X¹³ is a hydrogen atom or a C₆₋₁₀aryl radical, and m denotes a value from 1 to 4,

salts of isoindoline pigments of formula

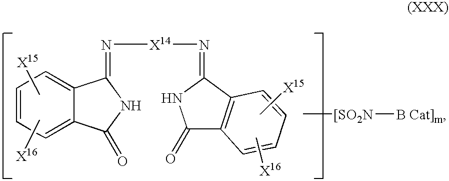

wherein X¹⁴ is the radical of an aromatic or heteroaromatic diamine, such as

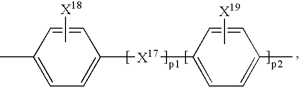

wherein p1 and p2 are 0 or 1, X¹⁸ and X¹⁹ are a hydrogen atom, a C₁₋₄alkyl radical, a C₁₋₄alkoxy radical or a chlorine atom, X¹⁷ is a group —CH₂—, —CH═CH— or —N═N—, X¹⁵ and X¹⁶ are a hydrogen atom, a C₁₋₄alkyl radical, a C₁₋₄-alkoxy radical, a nitro group or a chlorine atom and m denotes a value from 1 to 3, and B and Cat are as defined above.

Preferred colorants are:

pyrrolo[3,4-c]pyrrole derivatives of formula

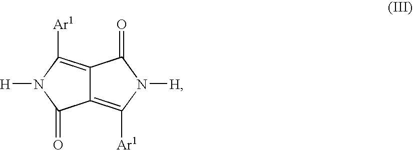

wherein Art is a group of formula

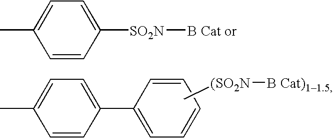

phthalocyanine derivatives of formula

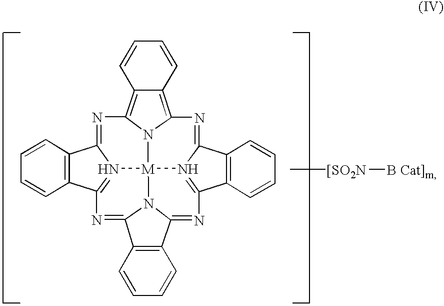

wherein M is Cu(II) or Zn(II), and m denotes a value from 3 to 5,

indanthrone derivatives of formula

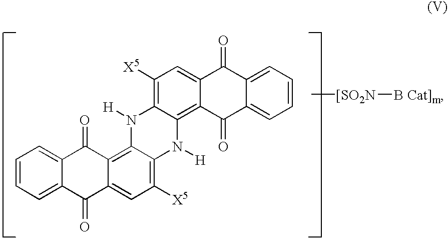

wherein X⁵ is a hydrogen or chlorine atom and m denotes a value from 2 to 4, and

quinacridone derivatives of formula

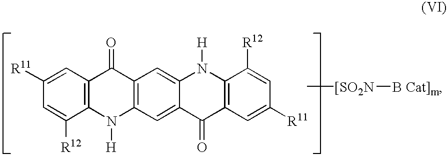

wherein R¹¹ and R¹² are each independently of the other hydrogen, a chlorine atom or a methyl group, m denotes a value from 1 to 4 and

B is a group —(CH₂)ₑ-E or

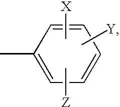

wherein e is an integer from 1 to 6, especially 2 or 3, E is a hydrogen atom or a group —OH, —OCat, —SH, —SCat, —OR¹, —SR², —NR⁵R⁶ or —C(O)OR³, and X, Y and Z are each independently of the others selected from a hydrogen atom and a group —OH, —OCat, —SH, —SCat, —OR¹, —SR², —NR⁵R⁶ and —C(O)OR³, wherein R¹, R² and R³ are each independently of the others a C₁₋₄alkyl radical, especially methyl or ethyl, and R⁵ and R⁶ denote a radical —(CH₂)60H, wherein o is an integer from 2 to 6, especially 2 or 3, and Cat is a sodium or potassium cation or unsubstituted ammonium or an ammonium cation described hereinbefore as preferred, especially tetramethylammonium; tetraethylammonium or

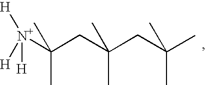

mono-, di- or triethanolammonium or mono-, di- or tri-isopropanolammonium, N-methyl-N-ethanolammonium or 2-, 3- or 4-hydroxyphenylammonium.

In the above formulae III to VI and X to XXX one to three groups —SO₂NBCat can be replaced by groups —SO₂NHB with the proviso that at least one group —SO₂NBCat is present. Examples of compounds modified in such a way are sulfonamide salts of the following formulae (IVa), (Va) and (Xa) and of formula (IVb) below:

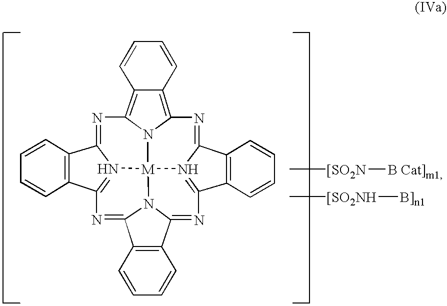

wherein M, Cat and B are as defined in formula IV, m1 and n1 denote a value from 1 to 4, especially 1 to 3, wherein the sum of m1 and n1 being 3 to 5, especially 4,

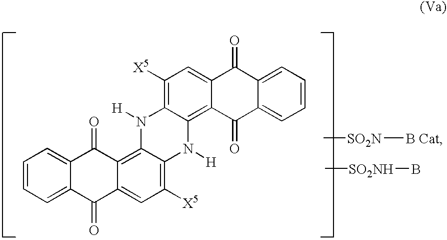

wherein B and Cat are as defined in formula V, and

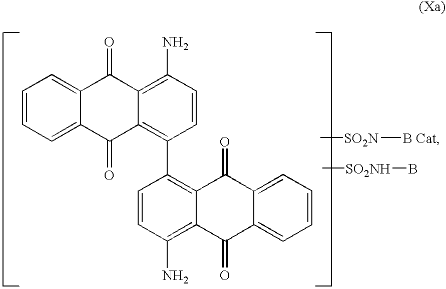

wherein B and Cat are as defined in formula X.

Sulfonamide derivatives in which —SO₂NHB/—SO₂NB is either —SO₂NH₂/—SO₂NH or a mixture of —SO₂NH₂/—SO₂NH and —SO₂NHB/—SO₂NB are especially suitable for the pigmenting of aluminium. Examples of such derivatives are phthalocyanine derivatives of formula

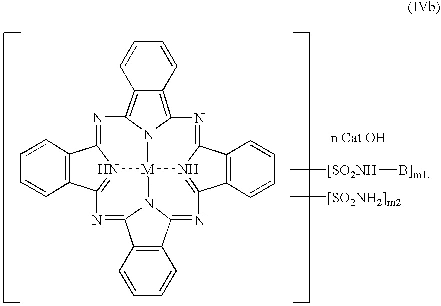

wherein M is Cu(II) or Zn(II), m1 denotes a value from 0 to 3, m2 denotes a value from 1 to 4, the sum of m1 and m2 being 3 to 5, especially 4, n denotes a value from 1 to 8, B is a group —(CH₂)ₑ-E or

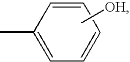

wherein e is an integer 2 or 3, E is a hydrogen atom, a group —OH, —SH, —OR¹, —SR², —NR⁵R⁶ or —C(O)OR³, and Cat is a sodium-or potassium cation or unsubstituted ammonium or an ammonium cation described hereinbefore as preferred.

The colorants are derived especially from C.I. Pigment Yellow 138, 139, 185, C.I. Pigment Brown 38, C.I. Pigment Orange 66, 69, C.I. Pigment Red 260; C.I. Pigment Red 123, 149, 178, 179, 190, 224, C.I. Pigment Violet 29, C.I. Pigment Black 31, 32; C.I. Pigment Blue 15:6; C.I. Pigment Violet 19, C.I. Pigment Red 122, 192, 202, 207 and 209; C.I. Pigment Red 254, C.I. Pigment Red 255, C.I. Pigment Red 264, C.I. Pigment Red 272, C.I. Pigment Orange 71, C.I. Pigment Orange 73; C.I. Pigment Blue 60 and 64; C.I. Pigment Violet 29, C.I. Pigment Red 123, 179, 190 or C.I. Pigment Violet 23 or 37.

Especially preferred as colorants of formula I are the compounds A1, A2, B1 to B5, C1 to C8, D1 to D55, E1 to E8, F1 and G1 to G3 listed in the Examples, compounds D4, D39, D53 and E1 to E8 being especially suitable for the pigmenting of wood, whilst compounds D54 and D55 yield very good results in the coloration of aluminium. For shade matching or to produce intermediate colours, it is also possible to use mixtures of colorants of formula I and/or II for the coloration. To produce colour effects, it is possible for a plurality of colorants of formula I or II to be deposited in succession into the pores of the porous materials.

Preferably, the colorants according to the invention do not contain deprotonatable carboxylic acid groups or sulfonic acid groups or benzimidazole radicals.

The colorants of formulae I and II according to the invention can be obtained from the corresponding sulfonamides by reaction with alkali metal hydroxides or ammonium hydroxides or amines. The sulfonamides that are used as starting materials are either known (see e.g. GB-A-1 198 501, U.S. Pat. No. 4,234,486, U.S. Pat. No. 6,066,203 etc.) or can be prepared according to known processes (see e.g. U.S. Pat. No. 6,066,203, 3rd column, lines 36 to 50). In the case of the colorants of formula I, the pigment is advantageously reacted with chlorosulfonic acid/thionyl chloride to form the pigment sulfonyl chloride, which is then reacted with the corresponding amine to form the sulfonamide.

The colorants of formula II can be obtained, for example, by nitrating a starting pigment, reducing the resulting nitro groups and reacting the resulting amino groups with the corresponding sulfonic acid chloride, or by using processes known per se to introduce aminomethyl groups into the starting pigments (see EP-A-311 562) and to react the amino groups with the corresponding sulfonic acid chloride.

The present invention relates also to the use of the colorants of the general formulae I and II for the pigmenting of porous materials, especially for the pigmenting of wood and anodised aluminium in the pores, and in inkjet printing.

The colorants according to the invention can be used individually or in mixtures with other colorants or, for example, dyes customary for the application in question.

The colorants of the invention are generally used in an amount effective for pigmenting, that is to say in an amount that is sufficient to bring about a colour difference ΔE* (CIE-L*a*b*)>2 when the pigmented material is compared with the unpigmented material using standard light type D₆₅ at an observation angle of 100. The amount is preferably from 0.01 to 30% by weight, especially from 0.1 to 15% by weight, based on the weight of the pigmented material.

The porous materials may be of natural or synthetic origin, and may be mineral or organic. Examples of porous materials are porous metal oxides, such as the oxides of elements of groups 2, 3, 4, 12, 13 and 14 of the Periodic Table, for example oxides of aluminium, silicon, magnesium and mixtures thereof, especially anodised light metals, especially aluminium, or alloys thereof, and porous synthetic materials, for example porous polyamide fillers, especially polyamide-12, polyamide-6 or co-polyamide-6/12 fillers, the manufacture of which is described in U.S. Pat. No. 4,831,061, FR-A-2 619 385 and EP-A-303 530 and which are sold by Atofina under the trade name Orgasol®. Further examples of porous materials are chalk, pumice, calcined clay, unglazed ceramics, gypsum, concrete, kieselguhr, silica gel, zeolites, wood, paper, leather, imitation leather and hair. The colorants according to the invention are suitable especially for pigmenting wood and anodised aluminium.

The principle according to the invention will now be described taking the example of wood and aluminium. It is clear, however, that with slight modifications the principle can be transferred to any other desired porous materials.

Wood is especially any kind of hard or soft wood, for example obeche, ash, birch, poplar, pine, spruce, fir, tulip tree, maple, bird's eye maple, sycamore, oak, beech, mahogany, myrtle, anigre, tay (koto), mappa burl, elm, zebrano, carbalho, vavona or daniela. The methods and conditions for treating wood and wood products are known from the specialist literature, to which reference is expressly made herein. For example, the methods and conditions for treatment with solutions are described in detail in Ullmann's Encyclopedia of Industrial Chemistry, Vol. A28, 305-393 (5th edition 1996) and in KirkOthmer Encyclopedia of Chemical Technology, Vol. 24, 579-611 (3rd edition 1978). The application temperature can be elevated but it is advantageous to keep it sufficiently low that decomposition of the colorant does not occur, or occurs only negligibly, during the minimum time required for application. Where appropriate, it is also possible to add to the colorant solution further substances known for the treatment of the material, for example fungicides, antibiotics, flame retardants or moisture repellants.

If, when pigmenting wood, the colorants are used in admixture, then the components of the mixture are preferably components, the colour of which in the pigmentary form is red, yellow, blue, green, brown or black. Brown shades of an especially natural appearance can be produced therefrom. Any colorants added thereto are also preferably red, yellow, blue, green, brown or black.

According to the invention water is used as solvent. Where appropriate, co-solvents may be used, such as alcohol, for example ethanol or propanol, ethers, for example diethyl ether or methoxypropanol, or ketones, for example acetone or methyl ethyl ketone, the amount thereof generally not exceeding 15% by weight, preferably not exceeding 10% by weight. It is especially preferred to use water alone as solvent.

The concentration of the colorant in water is customarily from 0.01% by weight to about 99% of the saturation concentration, although in some cases it is possible to use supersaturated solutions without premature precipitation of the salt. For many colorants, the optimum concentration is about from 0.05 to 10% by weight, often about from 0.1 to 5% by weight, colorant, based on water. The solution of the compounds of formula (I) generally has a pH of from 6 to 11. The pH will depend, inter alia, upon the material to be coloured; for example, for wood it is preferably from 8 to 10, and for aluminium preferably from 6 to 8.

The pH of the aqueous solutions of the sulfonamide salts can be adjusted by specific selection of the following parameters:

nature of groups B and B′,

number of groups [SO₂—N—B] and/or [N—SO₂—B] and nature of cations Cat.

For example, compounds D17, D46, D54 and D55 have a pH of 6 to 7 and are accordingly especially suitable for pigmenting aluminium.

In order to obtain a desired pH within the range from 6 to 11, there may also be used, for example, buffer solutions based on borax, Tris buffer [tris(hydroxymethyl)aminomethane], NaHCO₃, KH₂PO₄ and Na₂HPO₄.

Coloration a) is effected preferably at elevated temperature, for example at from 40 to 160° C. The temperature during coloration is especially from 60 to 140° C., more especially from 80 to 120° C. Coloration is then optionally followed by drying at from 40 to 160° C.

The conversion of the colorant to its pigmentary form is then effected by conversion of the sulfonamide salt groups to sulfonamide groups. This is effected, unless the substrate to be pigmented is itself sufficiently acidic, by the addition of acid. Suitable acids are in principle any organic and inorganic acids. Preference is given to the use of organic C₁₋₆-carboxylic acids, examples of which include formic acid, acetic acid, propionic acid, pivalic acid, oxalic acid, malonic acid, succinic acid and citric acid. The treatment with acid is preferably effected at room temperature. The concentration of the acid will be governed by the number of sulfonamide groups present in the salt form. Advantageously the acid is used in excess.

The salts of sulfonamides according to the invention are suitable especially for the pigmenting of composite wood materials that are to be processed only after being coloured. Thin wooden panels through which the colorant has fully penetrated are stuck together and shaped and then cut at a wide variety of angles, yielding artistic effects as a result of the grain. Such materials can be used especially in the production of design articles or for decorative purposes. The demands in terms of fastness to light and penetration are substantially higher in this application than in customary wood veneers. Very homogeneous penetration of the individual wooden panels is especially important, even in the case of relatively large thicknesses, since the core thereof will be revealed by artistic cutting. Especially good results in that respect are obtained with compounds A1, B3 and D12. Compound A1 in the full tone exhibits, after 20 hours' weathering, stability that is approximately 4 times greater than that of colorants C.I. Acid Red 194 and 361 and in the pastel tone, after 50 hours' weathering, exhibits a ΔE* that is twice as low as that of colorants C.I. Acid Red 194 and 361.

After 120 hours' weathering, in the full tone compound B3 exhibits a ΔE* that is approximately four times lower than C.I. Acid Red 194 and 361 and in the pastel tone, after 20 hours' weathering, exhibits a ΔE* that is approximately twice as low as that of C.I. Acid Red 194 and 361. Penetration into the wood matrix is good. A point of interest is the colour shift of some compounds after application. For example, compound A1 results in a red coloration, whereas compound B4 results in a brownish-red coloration and compound B5 results in an orange-red coloration.

Compound D12 in the full tone, after 120 hours' weathering, exhibits a ΔE* that is approximately 3 to 4 times lower than that of C.I. Basic Blue 123 and C.I. Acid Blue 258 and, with the use of 10% methoxypropanol as co-solvent, exhibits acceptable penetration into the wood matrix. Special mention should be made in particular of the fastness to light of compound D12, which, after 600 hours' weathering, in the full tone exhibits a ΔE* of only 5. In the pastel tone compound D12, after 50 hours' weathering, exhibits a ΔE* that is approximately 3 to 4 times lower than that of C.I. Basic Blue 123 and C.I. Acid Blue 258.

The colorants according to the invention also yield very good results in inkjet printing. Compound D17 and compound D46 (2.5% by weight-strength ink solutions) exhibit good printing results in printing using a bubble-jet printer and also a piezo printer; special mention should be made, in particular, of the very good water-resistance.

Also in the pigmenting of anodised aluminium or alloys thereof, surprisingly, good results are obtained using the colorants according to the invention, that is to say in particular homogeneous coloration and very good fastness to light are obtained.

Especially suitable as aluminium alloys are those wherein aluminium predominates, especially alloys with magnesium, silicon, zinc and/or copper, for example Al/Mg, Al/Si, Al/Mg/Si, Al/Zn/Mg, Al/Cu/Mg and Al/Zn/Mg/Cu, preferably those wherein the aluminium content is at least 90% by weight; the magnesium content is preferably ≦6% by weight; the silicon content is preferably ≦6% by weight; the zinc content is preferably ≦10% by weight and the copper content is advantageously ≦2% by weight, especially ≦0.2% by weight.

The oxide layers formed on the metallic aluminium or on the aluminium alloys may have been produced by chemical oxidation or preferably galvanically by anodic oxidation. The anodic oxidation of the aluminium or of the aluminium alloy for the passivation and formation of a porous layer can be effected according to known methods using direct current and/or alternating current, and using suitable electrolyte baths, for example with the addition of sulfuric acid, oxalic acid, chromic acid, citric acid or combinations of oxalic acid and chromic acid or sulfuric acid and oxalic acid. Such methods of anodisation are known in the art: DS method (direct current; sulfuric acid), DSX method (direct current; sulfuric acid with the addition of oxalic acid), DX method (direct current; oxalic acid), DX method with the addition of chromic acid, AX method (alternating current; oxalic acid), AX-DX method (oxalic acid; first alternating current and then direct current), AS method (alternating current; sulfuric acid) and chromic acid method (direct current; chromic acid). The current voltages are generally in the range from 5 to 80 Volt, preferably from 8 to 50 Volt; the temperatures are generally in the range from 5 to 50° C.; the current density at the anode is generally in the range from 0.3 to 5 A/dm², preferably from 0.5 to 4 A/dm², in general current densities as low as ≦2 A/dm² being suitable for producing a porous oxide layer; at higher voltages and current densities, e.g. in the range from 100 to 150 Volt and ≧2 A/dm², especially from 2 to 3 A/dm², and at temperatures up to 80° C. it is possible to produce especially hard and fine-pored oxide layers, for example according to the “Ematal” method using oxalic acid in the presence of titanium and zirconium salts. In the production of oxide layers that are then coloured adsorptively electrolytically or directly using a colorant of formula (I), in accordance with a preferred procedure that is customary per se in practice the current voltage is in the range from 12 to 20 Volt and the current density is preferably from 1 to 2 A/dm². Such anodisation methods are generally known in the art and have also been described in detail in the specialist literature, e.g. in Ullmanns “Enzyklopadie der Technischen Chemie”, 4th edition, volume 12, pages 196 to 198, or in the Sandoz brochures “Sanodal®” (Sandoz AG, Basle, Switzerland, Publication No. 9083.00.89) or in “Ratgeber fur das Adsorptive Farben von Anodisiertem Aluminium” [Guide to the adsorptive colouring of anodised aluminium] (Sandoz, Publication No. 9122.00.80). The layer thickness of the porous oxide layer is advantageously in the range from 2 to 35 μm, preferably from 5 to 30 μm, especially from 15 to 25 μm. In order to colour the oxide layer using the colorants of formula I or II, it is possible to use colouring methods that are customary per se, especially adsorption methods (substantially without current voltage), the colorant solution being applied to the surface of the oxide, for example, by spraying or roller application (depending on the form of the substrate) or preferably by immersion of the article to be coloured in a dye bath. The coloration is effected advantageously at temperatures below the boiling point of the liquor, advantageously at temperatures in the range from 15 to 80° C., preferably in the range from 15 to 70° C., especially from 20 to 60° C. The pH of the dye liquor is in the range from acidic to weakly basic, generally in the pH range from 3 to 8, preference being given to weakly acidic to virtually neutral conditions, especially a pH range of from 4 to 6. The colorant concentration and the duration of coloration can vary very widely depending upon the substrate and desired tinctorial effect. For example, colorant concentrations in the range of from 0.01 to 20 g/l, advantageously from 0.1 to 10 g/l, especially from 0.2 to 2 g/l, are suitable. The duration of coloration is generally in the range from 30 seconds to 1 hour and is preferably from 5 to 40 minutes.

The colorations obtained in that manner can be subjected to hot and/or cold sealing according to customary methods, optionally using suitable additives, the colorations advantageously being rinsed with water before sealing.

Sealing can be carried out in one or two steps, for example at pH values of from 4.5 to 8 using metal salts or oxides (e.g. nickel acetate or cobalt acetate) or using chromates. Moreover, as described in DE-A-3327191, sealing can be carried out using organic sealing agents, for example organic phosphonates and diphosphonates or water-soluble (cyclo)aliphatic polycarboxylic acids or aromatic ortho-hydroxycarboxylic acids at pH values in the range from 4.5 to 8.

For cold sealing it is possible to use especially nickel or cobalt salts in combination with alkali metal fluorides, such as NaF. According to the invention, it is possible, e.g. as described in EP-A-1087038, to carry out cold sealing using a sealing agent containing nickel ions Ni²⁺ and fluoride ions F′. Where appropriate, the sealing agents may comprise, for example, substrate- and/or coloration-stipulated sealing auxiliaries, for example cobalt compounds, in small amounts of up to 10% by weight. The sealing agents can be used together with further adjuvants, such as (anionic) surfactants, especially sulfo-group-containing surfactants, preferably condensation products of sulfo-group-containing aromatic compounds with formaldehyde, for example condensation products of sulfonated naphthalene or/and sulfonated phenols with formaldehyde to form oligomeric condensation products having a surfactant character, and/or anti-smut agents (see e.g. DE-A-3900169 or DE-C-3327191), which contain, for example, salts of organic acids and non-ionic surfactants, for example P3-almeco seal® 1 (Henkel). The cold sealing is generally carried out at temperatures below 45° C., preferably in the range from 18 to 40° C., especially from 20 to 40° C. The Ni²⁺ concentration in the sealing bath is advantageously in the range from 0.05 to 10 g/l, preferably in the range from 0.1 to 5 g/l. The pH value of the sealing bath is, for example, in the range from acidic to weakly basic, advantageously in the pH range from 4.5 to 8. The sealing time will depend on the layer thickness and is, for example, from 0.4 to 2 minutes, preferably from 0.6 to 1.2 minutes, per μm thickness of the oxide layer of the substrate, sealing advantageously being carried out for from 5 to 60 minutes, preferably from 10 to 30 minutes. For the preferred oxide layers that are at least 15 μm thick, preferably from 15 to 30 μm thick, which are especially suitable for external architectural components, a sealing time of from 10 to 30 minutes is suitable.

Hot treatment with water is advantageously carried out in a temperature range of from: 80° C. to boiling temperature, preferably from 90 to 100° C., or also with steam at temperatures of from 95 to 150° C. as appropriate under pressure, for example at an overpressure in the range of from 1 to 4 bar. The duration of after-sealing with water is generally in the range of from 15 to 60 minutes.

It may be advantageous to carry out two-step sealing, wherein in the first step cold sealing is carried out using at least one sealing agent, such as nickel acetate, optionally in the presence of a anti-smut agent, such as P3-almeco seal® 1 (Henkel), in deionised water, and in the second step hot after-sealing is carried out in deionised water.

In particular, two-step sealing is used in which in the first step cold sealing is carried out at about 40° C. in deionised water using from 0.1 to 5 g/l, especially from 1.5 to 2.5 g/l, of nickel acetate in the presence of from 1 to 3 g/l of a anti-smut agent, such as P3-almeco seal® 1 (Henkel), for from 5 to 60 minutes, preferably from 10 to 30 minutes, and in the second step hot after-sealing is carried out in boiling, deionised water for from 15 to 60 minutes, especially from 30 to 45 minutes.

Treatment of the sealed aluminium substrates with a strongly inorganic acid, such as nitric acid, hydrochloric acid or phosphoric acid, can result in an increase in the fastness to light and/or a change in the colour of the pigmented aluminium substrates.

The colorants of formula I and/or II according to the invention are also suitable for colouring using plane-parallel flakes (effect pigments). Plane-parallel flakes are used as pigments in surface-coatings and printing inks and, in contrast to ground pigments, are distinguished by the fact that they can be made very thin. Since, after application of the surface-coating, they are oriented in such a manner that their plane surfaces extend parallel to the surface of the substrate, they produce (in contrast to ground pigments which reflect light more or less diffusely) a directed reflection of incident light.

A further embodiment of the present invention accordingly relates to coloured aluminium pigments that comprise platelet-shaped aluminium substrates coated with a metal oxide layer, the metal oxide layer comprising the pigments of formula I′ and/or II′ and the metals of the metal layer being selected from vanadium, titanium, zirconium, silicon, aluminium and boron.

The ratio of thickness to diameter of the flakes is referred to as the form factor and is generally from 1:50 to 1:500. Depending on the preparation process, the particle size distribution of the aluminium pigments is more or less statistical, having a d₅₀ of from 5 to 50 μm.

The amount of colorant is generally from 5 to 40% by weight and the amount of metal oxide is from 3 to 95% by weight, in each case based on the aluminium substrate.

The aluminium pigments can be obtained in analogy to a process described in DE-A-195 01 307, by producing the metal oxide layer by means of a sol-gel process by controlled hydrolysis of one or more metal acid esters in the presence of one or more of the colorants according to the invention, optionally in the presence of an organic solvent and optionally in the presence of a basic catalyst.

Suitable basic catalysts are, for example, amines, such as triethylamine, ethylenediamine, tributylamine, dimethylethanolamine and methoxypropylamine.

Suitable aluminium pigments are any customary aluminium pigments that can be used for decorative coatings and the oxidised coloured aluminium pigments described in DE-A-195 20 312. Preference is given to round aluminium flakes (so-called silver dollars).

The organic solvent is a water-miscible organic solvent, such as a C₁₋₄alcohol, especially isopropanol.

Suitable metal acid esters are selected from the group consisting of alkyl and aryl alcoholates, carboxylates and carboxyl-, alkyl- or aryl-substituted alkyl alcoholates or carboxylates of vanadium, titanium, zirconium, silicon, aluminium and boron. Preference is given to the use of triisopropyl aluminate, tetraisopropyl titanate, tetraisopropyl zirconate, tetraethyl orthosilicate and triethyl borate. It is also possible to use acetylacetonates and acetoacetylacetonates of the above-mentioned metals. Preferred examples of that kind of metal acid ester are zirconium, aluminium and titanium acetylacetonate and diisobutyloleyl acetoacetylaluminate or diisopropyloleyl acetoacetylacetonate and mixtures of metal acid esters, for example Dynasil® (made by Hüls), a mixed aluminium-silicon metal acid ester.

Moreover, the aluminium pigment can be prepared analogously to a process described in EP-A-0 380 073. A layer of an anodically oxidisable metal is applied to a carrier that has optionally been coated with a separating agent, which layer has a thickness corresponding to at least 500 nm and which is oxidised anodically in an electrolyte at a voltage of from 0.5 to 100 V. The porous metal oxide layer is then coloured using the colorants according to the invention and sealed. The separating agent is then dissolved in a suitable solvent, the aluminium pigment precipitating in the form of coarse flakes which can be further processed by separation of the solvent, drying and milling (see, for example, WO01/25500 A1).

The carrier that has been coated with an anodically oxidisable metal can be obtained according to known processes. It is advantageous to use carriers to which a thin metal layer has been applied by sputtering or by chemical methods or by vapour-deposition in vacuo. The layer thickness of the metal is advantageously so selected that the metal layer remaining after anodic oxidation is covered with a metal oxide layer of a thickness of at least 10 nm, preferably at least 100 nm. The layer thickness of the metal is generally from 500 nm to 5 μm, preferably from 1 μm to 2 μm.

Suitable electrolytes are known and are described, for example, in J. Elektrochem. Soc.: Electrochemical Science and Technology, 122,1, p. 32 (1975). There are suitable, for example, dilute aqueous solutions (e.g. up to 20% by weight) of inorganic acids or of carboxylic acids (sulfuric acid, phosphoric acid, chromic acid, formic acid, oxalic acid), of alkali metal salts of inorganic acids or of carboxylic acids (sodium sulfate, sodium bisulfate, sodium formate), and alkali metal hydroxides (KOH, NaOH).

The anodic oxidation can be carried out at a temperature of from 0 to 60° C. and preferably at room temperature. The voltage to be selected will depend substantially on the electrolyte used and is generally from 0.5 to 100 V. Electrolysis can be carried out using alternating current or preferably using direct current.

The carrier has a surface of metal, glass, enamel, ceramics or an organic material and can be of any desired shape, foils, films and plates being preferred. The carrier can be, for example, a glass, a mineral (quartz, sapphire, ruby, beryl or silicate), a ceramic material, silicon or a synthetic material (cellulose, polymethacrylate, polycarbonate, polyester, polyolefin, polystyrene).

The separating agent will be an inorganic separating agent, such as a separating agent that can be evaporated in vacuo, such as a chloride, borate, fluoride or hydroxide, or some other inorganic substance described, for example, in U.S. Pat. No. 5,156,720 and U.S. Pat. No. 3,123,489, or an organic separating agent, such as a surface-coating, sodium stearate, lithium stearate, magnesium stearate, aluminium stearate, a fatty alcohol or a wax alcohol of the type CₓH_{y}O wherein 15<C<30, a paraffin wax, a branched or unbranched fatty acid wherein C>15 or a thermoplastic polymer.

The metal layer is formed of aluminium itself or of an aluminium alloy with, for example, Mg or Zn. A preferred lower limit for the layer thickness is 500 nm. The upper limit for the layer thickness is a maximum of 5.0 μm. The thickness is preferably from 0.5 to 3.0 μm and especially from 1.0 to 2.0 μm.

The thickness of the oxide layer will depend substantially on the initial thickness of the metal layer. The oxide layer can be, for example, from 10 nm to 500 nm. Preferred layer thickness ranges are from 100 nm to 500 nm.

The diameter of the pores in the metal oxide layer will depend substantially on the preparation conditions during electrolysis; especially on the electrolyte used. The diameter can be, for example, from 2 nm to 500 nm.

The aluminium pigments according to the invention can be used to provide a special effect in paints, coatings, plastics, printing inks and cosmetic preparations.

The following Examples illustrate the invention.

### EXAMPLES

### Synthesis Example 1

### Synthesis of Compound A1

Sulfochlorination:

230 g of chlorosulfonic acid are placed in a 0.5 litre round-bottomed flask which is provided with a stirrer, thermometer and condenser. 45 g of C.I. Pigment Red 264 (0.1 mol) are introduced, in portions, at room temperature. The resulting solution is heated slowly to 140° C. and stirred for 4 hours. The solution is cooled and 85 g of thionyl chloride are slowly added dropwise at 75° C. Stirring is then carried out for a further 4 hours at reflux. At room temperature, the solution is poured onto 2.5 kg of ice, and the resulting suspension is filtered and washed thoroughly with water.

Amination:

122 g of ethanolamine (2 mol) are placed in a 1 litre round-bottomed flask and cooled to 0° C. by the addition of ice. The moist aqueous filter cake is introduced in portions, the temperature being maintained at 0° C. by the further addition of ice. The suspension is stirred for one hour at 0° C., for 14 hours at room temperature and for 1 hour at 80° C. The suspension is cooled and 350 g of 32% hydrochloric acid solution are added dropwise. At room temperature, the suspension is filtered and washed with 3% hydrochloric acid solution. Drying in vacuo at 80° C. yields 70 g of compound A1′.

The ‘H-NMR shows that compound A1’ is both a mixture of structural isomers and a mixture of two- to three-fold sulfochlorinated and amidated molecules (ratio: 25% three-fold, 75% twofold sulfochlorinated species).

Elemental analysis (theory): C, 57.33% (57.94%); H, 4.18% (4.36%); N, 7.65% (8.28%); S, 10.32% (9.98%).

Conversion of Compound A1′ into Compound A1

20 h of compound A1′ are suspended in 200 g of water in a 1 litre round-bottomed flask. At room temperature, 7.5 g of 30% sodium hydroxide solution are added dropwise. The resulting solution is stirred for 2 hours at 60° C. and filtered while warm, and the filtrate is concentrated by evaporation at a maximum of 80° C. and at reduced pressure. Drying in vacuo at 80° C. yields 21 g of compound A1.

Elemental analysis, based on a 1:3 mixture of twofold and threefold sulfochlorinated molecules (theory): C, 53.33% (54.10%); H, 3.78% (4.10%); N, 7.15% (7.73%); S, 9.72% (9.30%); Na, 6.98% (6.67%).

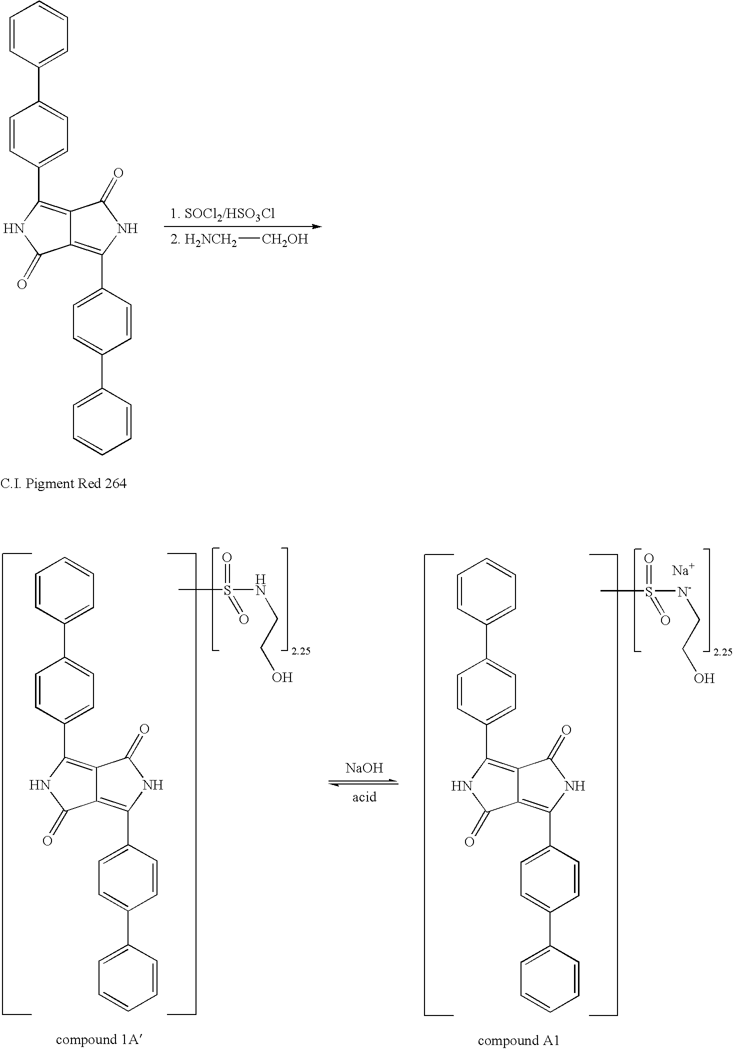

### Synthesis Example 2

### Synthesis of Compound A2

Compound A2 is prepared analogously to Synthesis Example 1.

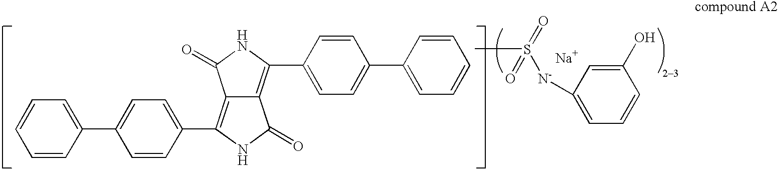

Compounds B1 to B5 are prepared analogously to Synthesis Example 1.

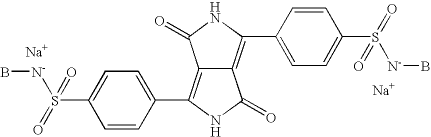

| Synthesis | | |
|---|---|---|
| Example | Compound | B |
| 3 | B1 | 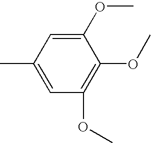 |
| 4 | B2 | 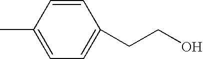 |
| 5 | B3 | 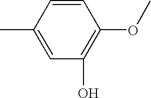 |
| 6 | B4 | 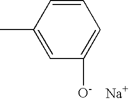 |
| 7 | B5 | 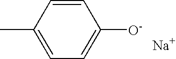 |

### Synthesis Examples 8 to 15

### Synthesis of Compounds C1 to C8

Compounds C1 to C8 are prepared analogously to Synthesis Example 1 starting from C.I. Pigment Blue 60.

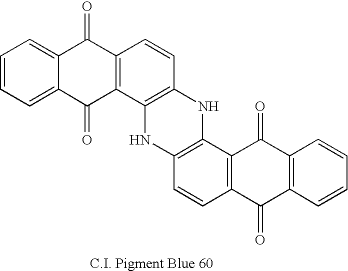

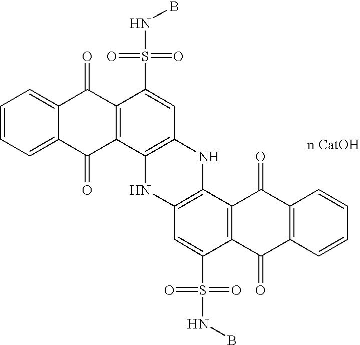

| Synthesis | | | |
|---|---|---|---|
| Example | Compound | B | n Cat |
| 8 | C1 | —CH₂CH₂OH | 2 Na⁺ |
| 9 | C2 | 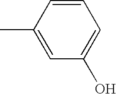 | 2 Na⁺ |
| 10 | C3 | ″ | 4 Na⁺ |
| 11 | C4 | H | Na⁺ |
| 12 | C5 | H | 2 Na⁺ |
| 13 | C6 | 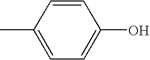 | Na⁺ |
| 14 | C7 | ″ | 2 Na⁺ |
| 15 | C8 | ″ | 3 Na⁺ |

### Synthesis Example 17

### Synthesis of Compound D1

10 g of compound D1′ (for preparation, see Example 1 of WO 98/45756) are suspended in 150 g of water in a 500 ml round-bottomed flask. At room temperature, 5.4 g of 30% sodium hydroxide solution are added dropwise. The resulting solution is stirred at 60° C. for 3 hours and filtered while warm, and the filtrate is concentrated at a maximum of 80° C. and at reduced pressure using a rotary evaporator. Drying in vacuo at 80° C. yields 21 g of compound D1.

Elemental analysis (theory): C, 41.37% (41.54%); H, 2.96% (2.79%); N, 14.14% (14.53%); Cu, 5.36 (5.49%); Na, 8.26% (7.95%); S, 10.91% (11.09%).

Compounds D2 to D55 are prepared analogously to Synthesis Example 17.

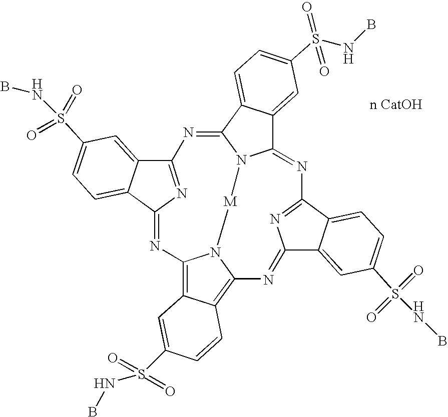

| Synthesis | | | | |
|---|---|---|---|---|
| Example | Compound | B | M | n Cat |
| 17 | D1 | —(CH₂)₂—OH | Cu | 4 Na⁺ |
| 18 | D2 | —(CH₂)₂—OH | Cu | 6 Na⁺ |
| 19 | D3 | —(CH₂)₂—OH | Cu | 8 Na⁺ |
| 20 | D4 | —(CH₂)₂—OH | Cu | 4 Li⁺ |
| 21 | D5 | —(CH₂)₂—OH | Cu | 8 Li⁺ |
| 22 | D6 | —(CH₂)₂—OH | Cu | 4 N(CH₃)₄⁺ |
| 23 | D7 | 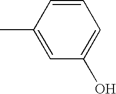 | Cu | 4 Na⁺ |
| 24 | D8 | ″ | Cu | 8 Na⁺ |
| 25 | D9 | 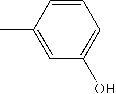 | Cu | 4 N(CH₃)₄⁺ |
| 26 | D10 | ″ | Cu | 8 N(CH₃)₄⁺ |
| 27 | D11 | 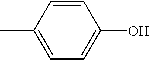 | Cu | 4 Na⁺ |
| 28 | D12 | ″ | Cu | 8 Na⁺ |
| 29 | D13 | 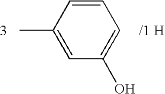 | Cu | 7 Na⁺ |
| 30 | D14 | 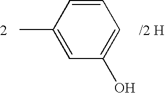 | Cu | 3 Na⁺ |
| 31 | D15 | ″ | Cu | 4 Na⁺ |
| 32 | D16 | ″ | Cu | 6 Na⁺ |
| 33 | D17 | 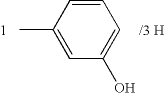 | Cu | 3 Na⁺ |
| 34 | D18 | ″ | Cu | 4 Na⁺ |
| 35 | D19 | ″ | Cu | 5 Na⁺ |
| 36 | D20 | 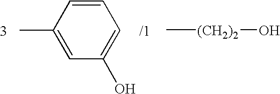 | Cu | 6 Na⁺ |
| 37 | D21 | ″ | Cu | 8 Na⁺ |
| 38 | D22 | 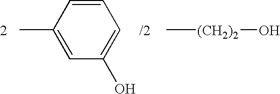 | Cu | 6 Na⁺ |
| 39 | D23 | ″ | Cu | 8 Na⁺ |
| 40 | D24 | 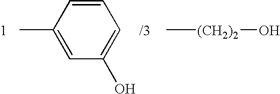 | Cu | 4 Na⁺ |
| 41 | D25 | ″ | Cu | 6 Na⁺ |
| 42 | D26 | ″ | Cu | 8 Na⁺ |
| 43 | D27 | 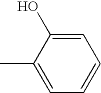 | Cu | 3 Na⁺ |
| 44 | D28 | ″ | Cu | 4 Na⁺ |
| 45 | D29 | ″ | Cu | 6 Na⁺ |
| 46 | D30 | ″ | Cu | 8 Na⁺ |
| 47 | D31 | —(CH₂)₃—OH | Cu | 4 Na⁺ |
| 48 | D32 | —(CH₂)₃—OH | Cu | 6 Na⁺ |
| 49 | D33 | —(CH₂)₃—OH | Cu | 8 Na⁺ |
| 50 | D34 | 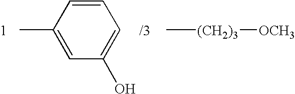 | Cu | 4 Na⁺ |
| 51 | D35 | ″ | Cu | 5 Na⁺ |
| 52 | D36 | 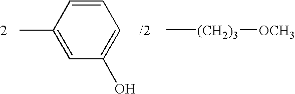 | Cu | 5 Na⁺ |
| 53 | D37 | ″ | Cu | 6 Na⁺ |
| 54 | D38 | 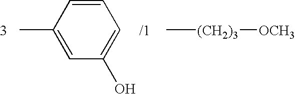 | Cu | 6 Na⁺ |
| 55 | D39 | ″ | Cu | 7 Na⁺ |
| 56 | D40 | —(CH₂)₂—OH | Zn | 4 Na⁺ |
| 57 | D41 | —(CH₂)₂—OH | Zn | 8 Na⁺ |
| 58 | D42 | 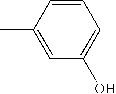 | Zn | 8 Na⁺ |
| 59 | D43 | 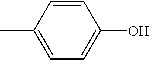 | Zn | 4 Na⁺ |
| 60 | D44 | ″ | Zn | 8 Na⁺ |
| 61 | D45 | 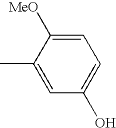 | Zn | 8 Na⁺ |
| 62 | D46 | H | Zn | 2 Na⁺ |
| 63 | D47 | H | Zn | 3 Na⁺ |
| 64 | D48 | H | Zn | 4 Na⁺ |
| 65 | D49 | 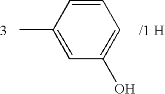 | Zn | 4 Na⁺ |
| 66 | D50 | ″ | Zn | 7 Na⁺ |
| 67 | D51 | 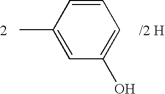 | Zn | 4 Na⁺ |
| 68 | D52 | ″ | Zn | 6 Na⁺ |
| 69 | D53 | —(CH₂)₂OH | Cu | 8 NH₄⁺ |
| 70 | D54 | H | Cu | 1 Na⁺ |
| 71 | D55 | 2 —(CH₂)₂OH/2 H | Cu | 1 Na⁺ |

### Synthesis Example 33

### Synthesis of Compound D17

In a 750 ml sulfonating flask, 2.03 g of 3-aminophenol (0.026 mol) and 5.31 g of a 25% ammonium hydroxide solution (0.078 mol) are introduced into a mixture of 9.4 g of water and 16.6 g of methanol and cooled to 0° C. by the addition of ice. 0.026 mol of copper phthalocyaninetetrasulfonic acid chloride is introduced in portions, the temperature being maintained at 0° C. by further addition of ice. The suspension is stirred for 30 minutes at 0° C. and for 14 hours at room temperature. The suspension is cooled and 10 g of 32% hydrochloric acid solution are added dropwise. At room temperature, the suspension is filtered and washed with 3% hydrochloric acid solution. Drying in vacuo at 70° C. yields 23.7 g of the sulfonamide D8′.

2 g of D8′ are suspended in 200 g of water in a 500 ml round-bottomed flask. 0.8 g of 30% sodium hydroxide solution is added dropwise at room temperature. The resulting solution is stirred at 60° C. for 2 hours and filtered while warm, and the filtrate is concentrated by evaporation at a maximum of 80° C. and at reduced pressure. Drying in vacuo at 80° C. yields 2.1 g of compound D17, which exhibits a pH value of 7.

### Synthesis Example 62

### Synthesis of Compound D46

Compound D46 is prepared analogously to Synthesis Example 17. Two equivalents of sodium hydroxide are used to form the salt in the last step. Only some of the amide functions are converted to a salt in order to establish a suitable pH value for the pigmenting of aluminium (see Application Example 7).

### Synthesis Example 72

### Synthesis of Compound E1

44.8 g of chlorosulfonic acid are introduced into a 250 ml sulfonating flask that is provided with a stirrer, thermometer and condenser. 10.0 g of 4,4′-diamino-1,1′-bisanthraquinone-3,3′-disodium sulfonate (0.0154 mol) are added in portions at room temperature. The resulting red solution is heated slowly to 40° C. and 13.2 g of thionyl chloride (0.11 mol) are added dropwise with stirring. The mixture is then heated to 70° C. and maintained at that temperature for 2 hours. After the solution has cooled, it is poured into a mixture of 50 g of water and 190 g of ice and the resulting suspension is filtered and washed thoroughly with water. The moist filter cake is added in portions, at 0° C., to a solution of 5.0 g of 3-aminophenol in a mixture of 8 g of water and 27 g of methanol. The suspension is stirred for one hour at 0° C., for 14 hours at room temperature and for 1 hour at 80° C. The suspension is cooled and 6 g of 32% hydrochloric acid solution are added dropwise. At room temperature, the suspension is filtered and washed with 3% hydrochloric acid solution. Drying in vacuo at 80° C. yields 10 g of the disulfonamide E1′.

2 g of the disulfonamide E1′ are suspended in 100 g of water in a 250 ml round-bottom flask. At room temperature, 0.67 g of 30% sodium hydroxide solution are added dropwise. The resulting solution is stirred at 60° C. for 2 hours and filtered while warm, and the filtrate is concentrated by evaporation at a maximum of 80° C. and under reduced pressure. Drying in vacuo at 80° C. yields 1.87 g of E1.

Elemental analysis (theory): C, 57.66% (57.83%); H, 3.29% (2.91%); N, 6.55% (6.74%); S, 7.73% (7.72%); Na, 5.97% (5.53%).

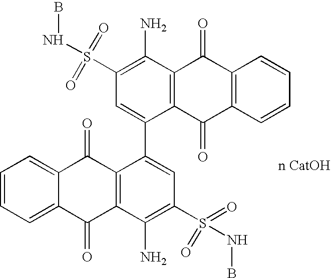

Compounds E2 to E8 are synthesised analogously to Synthesis Example 72.

| Synthesis | | | |
|---|---|---|---|
| Example | Compound | B | n Cat |
| 72 | E1 | 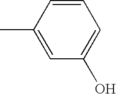 | 2 Na⁺ |
| 73 | E2 | ″ | 1 Na⁺ |
| 74 | E3 | ″ | 3 Na⁺ |
| 75 | E4 | —(CH₂)₂—OH | 1 Na⁺ |
| 76 | E5 | —(CH₂)₂—OH | 2 Na⁺ |
| 77 | E6 | H | 1 Na⁺ |
| 78 | E7 | H | 2 Na⁺ |
| 79 | E8 | 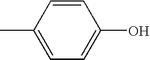 | 2 Na⁺ |

### Synthesis Example 80

### Synthesis of F1

Compound F1 is obtained analogously to Synthesis Example 62.

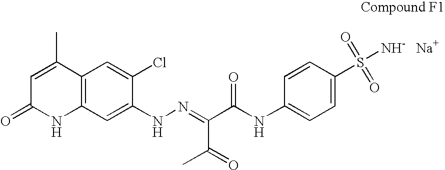

### Synthesis Examples 81 to 83

### Synthesis of G1 to G3

Compounds G1 to G3 are obtained analogously to Synthesis Example 62.

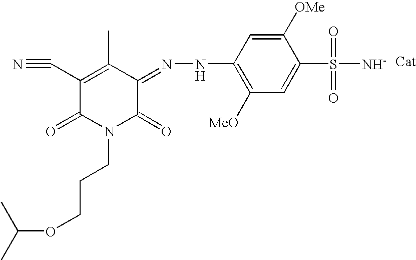

Compound G1

| Synthesis | | | |
|---|---|---|---|
| Example | Compound | B | Cat |
| 81 | G1 | H | NH₄⁺ |
| 82 | G2 | H | HO(CH₂)₂NH₃⁺ |
| 83 | G3 | H | Na⁺ |

Full Tone Coloration:

For full coloration, a 0.5-2% solution is used. The application time is 6 hours at 110° C. in an autoclave. Thereafter, the wood is dried for 30 minutes at 80° C., immersed in 5% citric acid at room temperature for 1 hour and finally dried at 130° C. for 30 minutes.

Pastel Tone Coloration:

For pastel tone coloration, a 0.05-0.2% solution is used. The application time is 6 hours at 110° C. in an autoclave. Thereafter, the wood is dried for 30 minutes at 80° C., immersed in 5% citric acid at room temperature for 1 hour and finally dried at 130° C. for 30 minutes.

Exposure Test in a Weathering Device (ATLAS WOM 6500W Xenon Radiation Device):

The coloured samples are mounted on frames (width 44 or 60 mm) and irradiated for up to 600 hours in the weathering device under the following parameters:

filter system: quartz internal filter/borosilicate external filter, corresponding to:

0.35 W/m² at 340 nm or 0.126 J/cm²/h

from 340 to 420 nm: 17-18 J/cm²/h

from 340 to 700 nm: 122-125 J/cm²¹ h

black-panel temperature: 63° C.

rel. humidity: 60%

Lightness (L), saturation (C) and hue (h) are measured using a spectrophotometer before irradiation and after 25, 50, 120, 240, 360, 480 and 600 hours' exposure. The corresponding colour differences and overall colour difference ΔE* are calculated therefrom.

### Application Example 1

### Pigmenting of Wood

1 to 5 pieces of dark obeche wood (115×30×1 mm) are immersed, in a sealed container (L=130 mm, diameter 36 mm), in a solution of 1.0% compound A1+0.15% NaHCO₃+0.1% Invadin in deionised water and are coloured at 110° C. for 6 hours with continuous rotation. 15 minutes' drying at 100° C. yields pieces of dark-red wood exhibiting homogeneous penetration, which are immersed for 1 hour in 5% citric acid+0.5% Invadin in water and then dried for five minutes at 160° C.

### Application Example 2

### Pigmenting of Wood

1 to 5 pieces of bleached obeche wood are immersed and coloured thoroughly, analogously to Application Example 1, for 6 hours at 110° C. in a solution of 0.05% compound A1+0.1% NaHCO₃+0.1% Invadin in deionised water. Drying for fifteen minutes at 100° C. yields pieces of light-red wood exhibiting homogeneous penetration.

### Application Example 3

### Pigmenting of Wood

1 to 5 pieces of dark obeche wood (115×30×1 mm) are immersed and coloured thoroughly, analogously to Application Example 1, for 6 hours at 110° C. in a solution of 0.55% compound D1+0.15% NaHCO₃+0.1% Invadin in deionised water. Drying for fifteen minutes at 100° C. yields pieces of dark-blue wood exhibiting good penetration, which are immersed for 1 hour in 5% citric acid+0.5% Invadin in water and then dried for 5 minutes at 160° C. The colorant is insoluble in water after the treatment (no bleeding).

### Application Example 4

### Pigmenting of Wood

1 to 5 pieces of dark obeche wood (115×30×1 mm) are immersed and coloured thoroughly, analogously to Application Example 1, for 6 hours at 110° C. in a solution of 0.5% compound F4+0.35% Na₂CO₃+0.1% Invadin in warm deionised water.

Drying for fifteen minutes at 100° C. yields pieces of yellow wood exhibiting homogeneous penetration, which are immersed for 1 hour in 5% citric acid+0.5% Invadin in water and then dried for five minutes at 160° C. The colorant is insoluble in water after the treatment (no bleeding).

### Application Example 5

### Inkjet Printing

Compound D9 (4 equivalents of tetramethylammonium hydroxide) and compound D10 (8 equivalents of tetramethylammomium hydroxide) are tested in inks (2.5% by weight strength solutions). They exhibit good printing results in printing using a bubble-jet printer and using a piezo printer. The chromaticity and the colour hue are within a range to be expected on the basis of the chromaticity and colour hue of the compounds not substituted by sulfonamide groups. The water-resistance is very good.

### Application Example 6

### Pigmenting of Wood

1 to 5 pieces of dark obeche wood (115×30×1 mm) are immersed and coloured thoroughly, analogously to Application Example 1, for 6 hours at 110° C. in a solution of 0.05% compound E1+0.15% Na₂CO₃+0.1% Invadin in warm deionised water. Drying for fifteen minutes at 100° C. yields pieces of yellow wood exhibiting homogeneous penetration, which are immersed for 1 hour in 5% citric acid+0.5% Invadin in water and then dried for five minutes at 160° C. The colorant is insoluble in water after the then dried for five minutes at 160° C. The colorant is insoluble in water after the treatment (no bleeding).

### Application Example 7

### Pigmenting of Anodised Aluminium

A degreased and deoxidised sheet of pure aluminium is oxidised anodically in an aqueous solution containing, in 100 parts, from 18 to 22 parts of sulfuric acid and from 1.2 to 7.5 parts of aluminium sulfate, at a temperature of from 18 to 20° C., at a voltage of from 15 to 16 Volt with direct current of a density of 1.5 A/dm², for from 30 to 40 minutes. An oxide layer of about from 18 to 20 μm thickness is formed having a porosity of 17%. After rinsing with water, the anodised aluminium sheet is coloured for 40 minutes at 60° C. in a solution consisting of 0.5 part of colorant D-54 in 100 parts of deionised water, the pH of which has been adjusted to 5.5 with acetic acid and sodium acetate. The alox layer is then sealed for 20 minutes at 40° C. in a solution of 2 g/litre of nickel acetate and 2 g/litre of P3-almeco seal® (Henkel) in deionised water and then sealed again for 40 minutes in boiling deionised water. The sample is then exposed in an Atlas-Weather-O-meter Ci 65 A. The colour difference ΔE resulting after 1500 hours is 5.0. Similar results are obtained with colorant D-55.

Compound A1 is tested in respect of full tone coloration and pastel tone coloration. The penetration is measured and the light stability is compared with the light stability of the standard dyes C.I. Acid Red 194 and 361. Compound A1 exhibits very good penetration and no bleeding. If the acid treatment is omitted during pigmenting with compound A1, the obeche veneers obtained in that manner exhibit a slight degree of bleeding. After 20 hours' weathering, compound A1 exhibits stability that is about 4 times greater than that of dyes C.I. Acid Red 194 and 361. Compound A1 not treated with acid exhibits slightly less stability. In the pastel tone, after 50 hours' weathering, compound A1 exhibits a ΔE* that is twice as low as that of dyes C.I. Acid Red 194 and 361.

Compounds B2 and B3 are tested in the full tone and in the pastel tone in comparison with standard dyes C.I. Acid Red 194 and 361. After 120 hours' weathering, compound B2 exhibits in the full tone a ΔE* that is approximately twice as low, and compound B3 exhibits a ΔE* that is approximately 4 times as low, as C.I. Acid Red 194 and 361. In the pastel tone, after 20 hours' weathering both compound B2 and B3 exhibit a ΔE* that is approximately twice as low as C.I. Acid Red 194 and 361. The penetration into the wood matrix is good for both compounds B2 and B3. A point of interest is the colour shift of some compounds after application. Compound A1 results in a red coloration, whereas compound B4 results in a brownish-red coloration and compound B5 results in an orange-red coloration.

The fastness to light of compounds D1, D8, D12 and D40 is compared in the full tone and in the pastel tone with the standard dyes C.I. Basic Blue 123 and C.I. Acid Blue 258. The copper phthalocyanine derivatives, compounds D8 and D12 in the full tone, after 120 hours' weathering, exhibit a ΔE* that is approximately 3 to 4 times lower than that of C.I. Basic Blue 123 and C.I. Acid Blue 258 and, using 10% methoxypropanol as cosolvent, exhibit acceptable penetration into the wood matrix. Special mention may be made, in particular, of the fastness to light of compound D8, which, after 600 hours' weathering, in the full tone exhibits a ΔE* of only 5. In the pastel tone compounds D8 and D12, after 50 hours' weathering, exhibit a ΔE* that is approximately 3 to 4 times lower than that of C.I. Basic Blue 123 and C.I. Acid Blue 258. The zinc phthalocyanine derivative (compound D40) is, in respect of fastness to light, slightly better than C.I. Basic Blue 123 and C.I. Acid Blue 258 and exhibits better penetration into the wood matrix than the copper phthalocyanine derivatives.

What is claimed is:

1. A colorant of the general formula 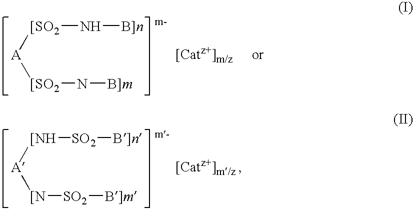
wherein n and n′ denote a value from 0 to 4,
m and m′ denote a value from 1 to 8, the sum of m+n and of m′ and n′ being less than or equal to 8,
z is an integer from 1 to 5, especially 1,
A is the radical of a chromophore of the series 1-aminoanthraquinone, anthraquinone, anthrapyrimidine, azo, azomethine, benzodifuranone, quinacridone, quinacridonequinone, quinophthalone, diketopyrrolopyrrole, dioxazine, flavanthrone, indanthrone, indigo, isoindoline, isoindolinone, isoviolanthrone, perinone, perylene, phthalocyanine, pyranthrone or thioindigo,
A′ is the radical of a chromophore that already contains one or more primary amino groups, such as 1-aminoanthraquinone, or A′ is one of the chromophore radicals listed under A modified with from 1 to 8, preferably with from 1 to 4, amino groups,
Cat is an alkali metal cation or an ammonium cation and
B and B′ are each independently of the other a branched or straight-chain C₁₋₈-alkyl, C₂₋₈alkenyl, C₂₋₈alkynyl, aryl, N-, O- or S-containing 5- or 6-membered heterocyclic ring, C₁₋₈alkylarylene, aryl-C₁₋₈alkylene or aryl-L-arylene radical, which may be substituted by one or more groups —OH, —OCat, —SH, —SCat, —OR¹, —SR², —C(O)OR³, —C(O)R⁴ and/or —NR⁵R⁶, it being possible for the C₁₋₈alkyl radical to be uninterrupted or interrupted one or more times by —O— or by —S—,
R¹, R², R³ and R⁴ are each independently of the others a C₁₋₈alkyl radical, a C₇₋₁₁-aralkyl radical or a C₆₋₁₂aryl radical and R⁴ can additionally be a hydrogen atom,
L is a bond —NR⁷, wherein R⁷ is a hydrogen atom or a C₁₋₄alkyl radical, or an —N═N— group, and R⁵ and R⁶ are each independently of the other a hydrogen atom, a C₁₋₈alkyl radical, a C₁₋₄alkoxy-C₁₋₄alkyl radical, a C₆₋₁₂aryl radical, a C₇₋₁₁aralkyl radical or a radical —(CH₂)ₒOH, wherein o is an integer from 2 to 6, and B can additionally be a hydrogen atom, it being possible for B and B′ within a chromophore A or A′ to have different substituent meanings.

2. A colorant according to claim 1, wherein B is selected from a hydrogen atom, 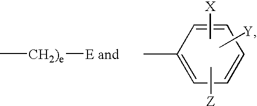
wherein e is an integer from 1 to 6, especially 2 or 3,
E is a hydrogen atom, a group —OH, —OCat, —SH, —SCat, —OR¹, —SR², —NR⁵R⁶ or —C(O)OR³, and X, Y and Z are each independently of the others selected from a hydrogen atom and a group —OH, —OCat, —SH, —SCat, —OR¹, —SR², —NR⁵R⁶ and —C(O)OR³, wherein R¹, R² and R³ are each independently of the others a C₁₋₄alkyl radical, especially methyl or ethyl, and R⁵ and R⁵ denote a radical —(CH₂)ₒOH, wherein o is an integer from 2 to 6, and Cat is a sodium or potassium cation, unsubstituted ammonium or an ammonium cation.

3. A colorant according to claim 1 or 2, wherein n is 0.

4. A colorant according to any one of claims 1 to 3, namely
a 1-aminoanthraquinone or anthraquinone derivative of formula 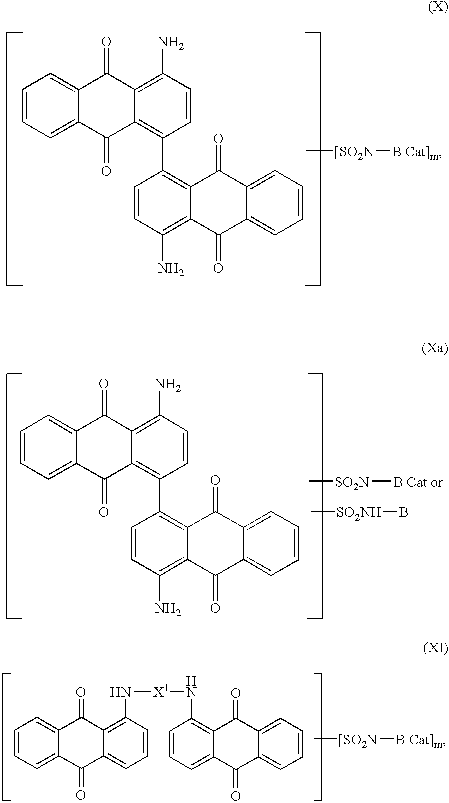
wherein X¹ is a group 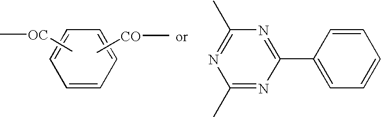
and m denotes a value from 1 to 4, especially from 2 to 3;
a quinacridone derivative of formula 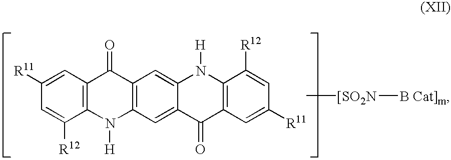
wherein R¹¹ and R¹² are each independently of the other hydrogen, halogen, C₁-C₂₄alkyl, C₁-C₆alkoxy or phenyl and m denotes a value from 1 to 4, especially from 2 to 3;
a pyrrolo[3,4-c]pyrrole derivative of formula 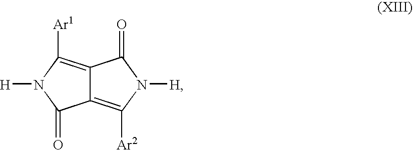
wherein Ar¹ and Ar² are each independently of the other a group of formula 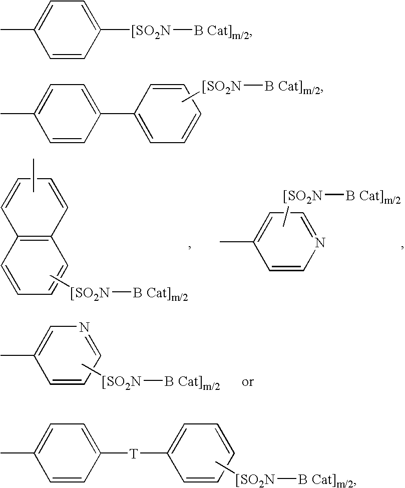
wherein T is —CH₂—, —CH(CH₃)—, —C(CH₃)₂—, —CH═N—, —N═N—, —O—, —S—, —SO—, —SO₂— or —NR¹³—, wherein R¹³ is hydrogen or C₁₋₆alkyl, especially methyl or ethyl, and m denotes a value from 1 to 4, especially from 2 to 3;
a dioxazine derivative of formula 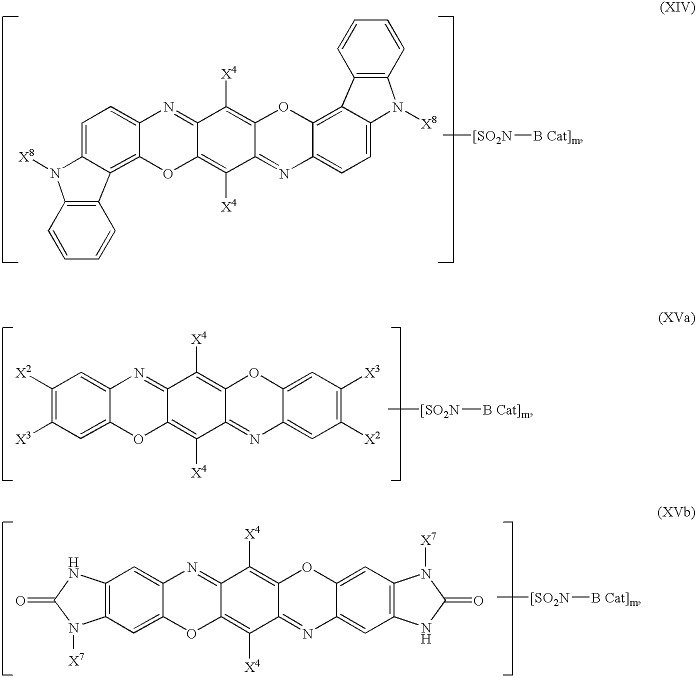
wherein X² is a C₁₋₄alkoxy radical, especially ethoxy, X³ is a C₁₋₄acylamino group, especially an acetylamino group, or a benzoylamino group and X⁴ is a chlorine atom or a radical NHC(O)CH₃, X⁷ is a hydrogen atom, a C₁₋₈alkyl radical, a substituted or unsubstituted phenyl, benzyl, benzanilide or naphthyl group, a C₅₋₇-cycloalkyl radical or a radical of formula 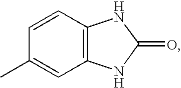
X⁸ is a hydrogen atom or a C₁₋₄alkyl radical and m denotes a value of from 1 to 4;
a flavanthrone derivative of formula 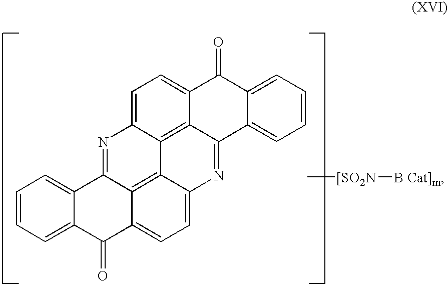
wherein m denotes a value from 1 to 4, especially from 2 to 3;
an indanthrone derivative of formula 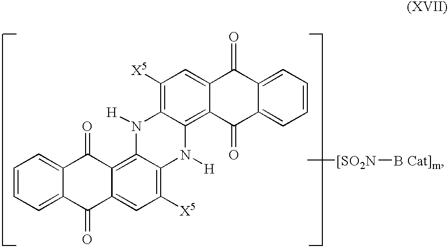
wherein X⁵ is a hydrogen or chlorine atom and m denotes a value from 1 to 4, preferably from 2 to 3;
an indigo derivative of formula 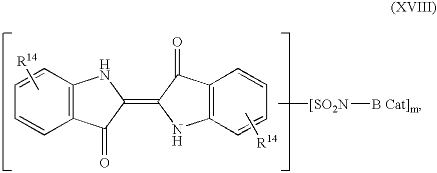
wherein R¹⁴ is hydrogen, CN, C₁₋₆alkyl, C₁₋₆alkoxy or halogen and m denotes a value from 1 to 3;
an isoviolanthrone derivative of formula: 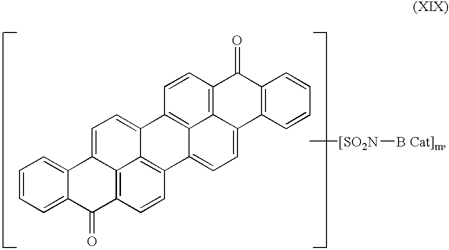
wherein m denotes a value from 1 to 4,
a perinone derivative of formula 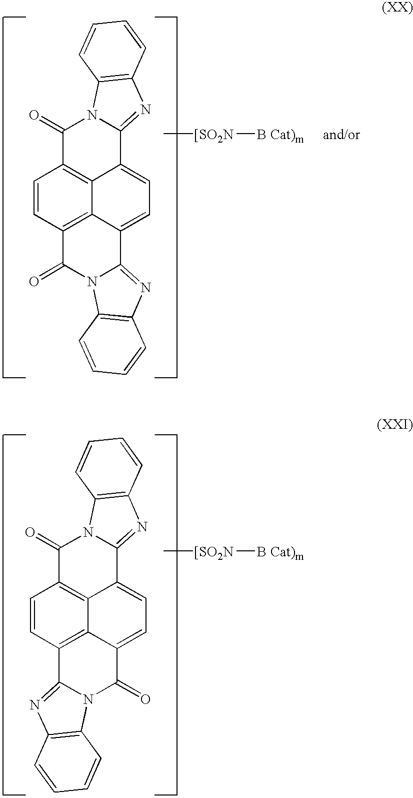
wherein m denotes a value from 1 to 4;
a perylene derivative of formula 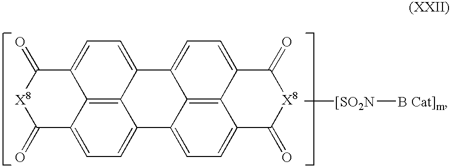
in which X⁶ is O or NR¹⁵, R¹⁵ being H, CH₃ or unsubstituted or substituted phenyl or C₇₋₁₁aralkyl, such as benzyl or 2-phenylethyl, and m denotes a value from 1 to 4, especially from 2 to 3, it being possible for the phenyl ring to be substituted by methyl, methoxy, ethoxy or by —N═N—Ph;
a phthalocyanine derivative of formula 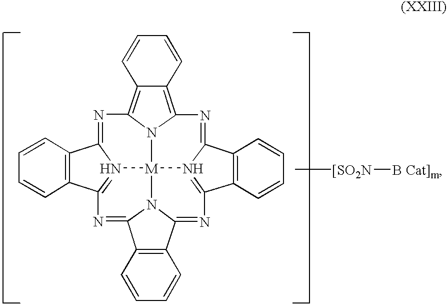
wherein M is H₂, a bivalent metal selected from the group Cu(II), Zn(II), Fe(II), Ni(II), Ru(II), Rh(II), Pd(II), Pt(II), Mn(II), Mg(II), Be(II), Ca(II), Ba(II), Cd(II), Hg(II), Sn(II), Co(II) and Pb(II), or a bivalent oxo metal selected from the group V(O), Mn(O) and TiO, and m denotes a value from 2 to 6, especially from 3 to 5;
a pyranthrone derivative of formula 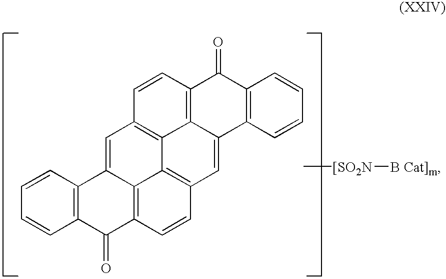
or a bromo-, chloro- or bromo- and chloro-halogenated derivative of the basic structure, for example the 2,10-dichloro, 4,6- or 6,14-dibromo derivative, wherein
m denotes a value from 2 to 4,
a thioindigo derivative of formula 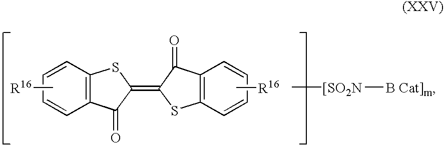
wherein R¹⁶ is hydrogen, CN, C₁₋₆alkyl, especially methyl, C₁₋₆alkoxy, especially methoxy, or halogen, especially chlorine, and m denotes a value from 1 to 3,
a monoazo yellow and orange derivative of formula 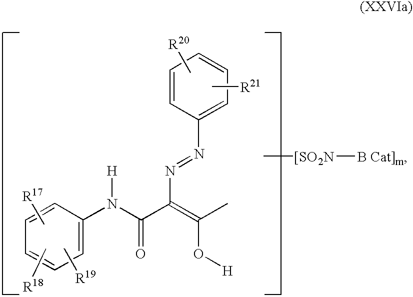
wherein m denotes a value from 1 to 4,
a diaryl yellow pigment derivative of formula 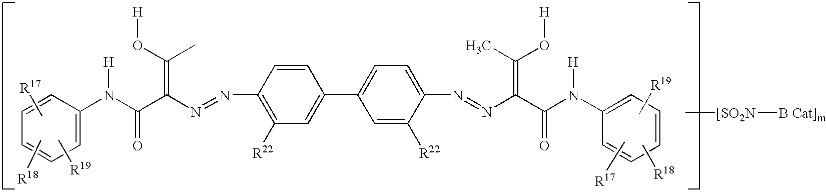
(XXVIb), wherein m denotes a value from 2 to 4,
a naphthol AS pigment derivative of formula 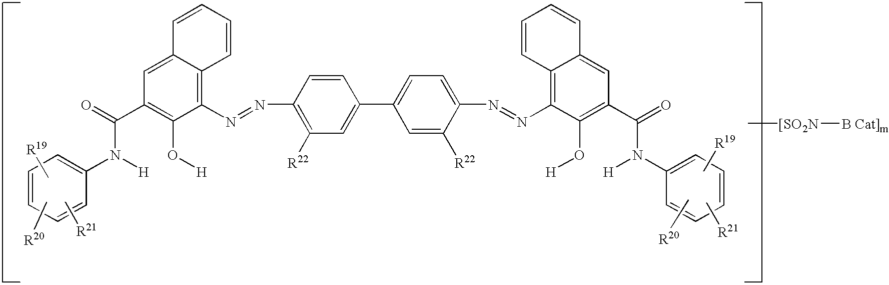
(XXVIc), wherein m denotes a value from 2 to 6,
R¹⁷ to R²¹ are each independently of the others a hydrogen atom, a halogen atom,
C₁₋₆alkyl, C₁₋₆alkoxy, a nitro group or an acetyl group and
R²² is a hydrogen atom, a halogen atom, C₁₋₆alkyl or C₁₋₆alkoxy,
a monoazoquinolone pigment of formula 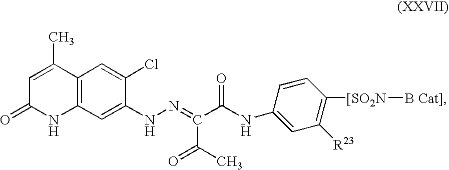
wherein R²³ is hydrogen, halogen, C₁₋₄alkyl, C₁₋₄alkoxycarbonyl, C₁₋₄alkylcarbonyl or C₁₋₄alkanoylamino, and
an azo pigment of formula 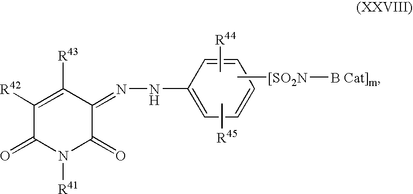
wherein
R⁴ is a hydrogen atom, a C₁₋₄alkyl radical, such as methyl or ethyl, or a perfluoro-C₁₋₄alkyl radical, such as trifluoromethyl, a hydroxy-C₁₋₄alkyl radical or a C₁₋₈alkyl radical interrupted one or more times by —O—, such as CH₂CH₂CH₂—O—CH(CH₃)₂, a C₆₋₁₂aryl radical, such as phenyl, or a C₇₋₁₂aralkyl radical, such as benzyl,
R⁴² is a hydrogen atom, or a cyano or carbonamide group,
R⁴³ is a hydrogen atom, a carboxylic acid group or a salt thereof or a C₁₋₄alkyl radical,
R⁴⁴ and R⁴⁵ denote a C₁₋₄alkyl radical, such as methyl or ethyl, a perfluoro-C₁₋₄alkyl radical, such as trifluoromethyl, a C₁₋₄alkoxy radical, such as methoxy or ethoxy, a nitro group, a halogen atom, such as chlorine, COOR⁴⁶, wherein R⁴⁶ is a C₁₋₄alkyl radical, a C₆₋₁₂alkyl radical that is unsubstituted or substituted, for example, by one or two chlorine atoms, such as phenyl or 1,4-dichlorophenyl, or a C₇₋₁₁aralkyl radical, such as benzyl, CONHR⁴⁷, wherein R⁴⁷ is a C₁₋₄alkyl radical, a C₆₋₁₂aryl radical, such as phenyl, or a C₇₋₁₁aralkyl radical, such as benzyl, and m denotes a value from 1 to 2,
an isoindoline pigment of formula 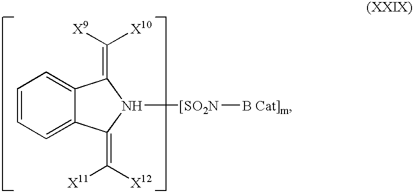
wherein
X⁹, X¹⁰, X¹¹ and X¹² are CN, CONH—C₁₋₈alkyl or CONH—C₆₋₁₂aryl or X⁹ and X¹⁰ and/or X¹¹ and X¹² are each members of a heterocyclic ring, such as 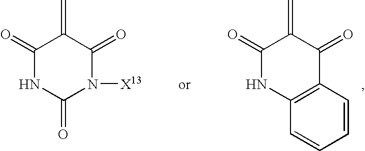
wherein X¹³ is a hydrogen atom or a C₆₋₁₂aryl radical, and m denotes a value from 1 to 4,
an isoindoline pigment of formula 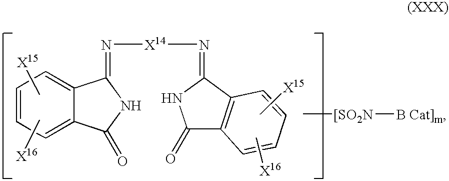
wherein X¹⁴ is the radical of an aromatic or heteroaromatic diamine and X¹⁵ and X¹⁶ are a hydrogen atom, a C₁₋₄alkyl radical, a C₁₋₄alkoxy radical, a nitro group or a chlorine atom and m denotes a value from 1 to 3, and
B and Cat are as defined in claim 1.

5. A colorant according to claim 4, namely
a pyrrolo[3,4-c]pyrrole derivative of formula 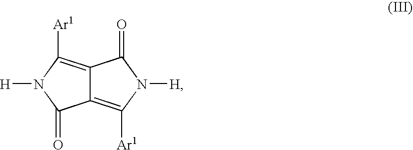
wherein Ar¹ is a group of formula 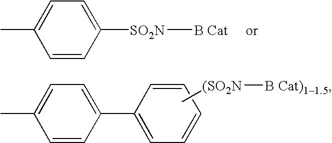
a phthalocyanine of formula 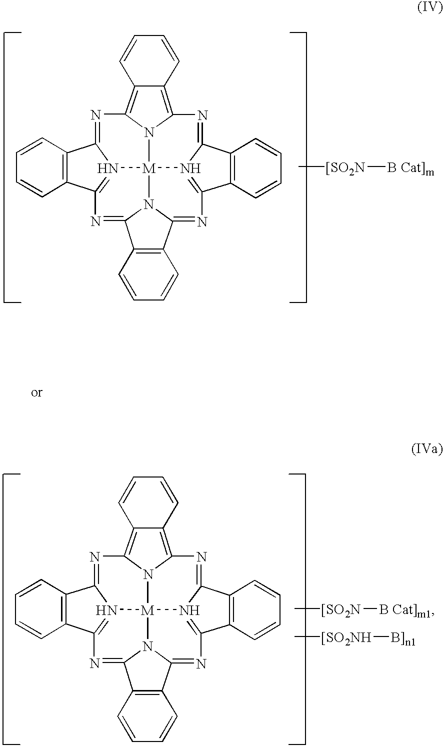
wherein M is Cu(II) or Zn(II) and m denotes a value from 3 to 5, m1 and n1 denote a value from 1 to 4, especially 1 to 3, wherein the sum of m1 and n1 being 3 to 5, especially 4,
an indanthrone of formula 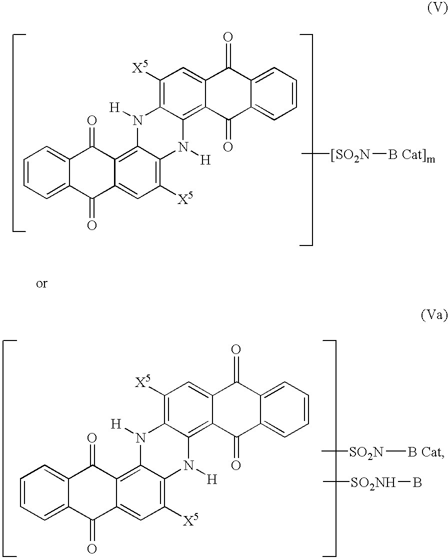
wherein X⁵ is a hydrogen or chlorine atom and m denotes a value from 2 to 4, or a quinacridone pigment of formula 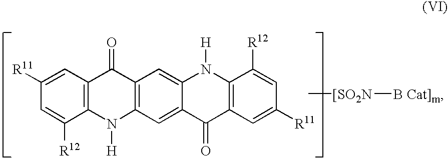
wherein R¹¹ and R¹² are each independently of the other hydrogen, a chlorine atom or a methyl group, m denotes a value from 1 to 4, and B is a group —(CH₂)ₑ-E or 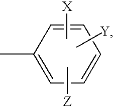
wherein e is an integer from 1 to 6, especially 2 or 3, E is a hydrogen atom or a group —OH, —OCat, —SH, —SCat, —OR¹, —SR², —NR⁵R⁶ or —C(O)OR³, and X, Y and Z are each independently of the others selected from a hydrogen atom and a group —OH, —OCat, —SH, —SCat, —OR¹, —SR², —NR⁵R⁶ and —C(O)OR³, wherein R¹, R² and R³ are each independently of the others a C₁₋₄alkyl radical, especially methyl or ethyl, and R⁵ and R⁶ denote a radical —(CH₂)ₒOH, wherein o is an integer from 2 to 6, especially 2 or 3, and Cat is a sodium or potassium cation or unsubstituted ammonium or an ammonium cation, such as tetramethylammonium, tetraethylammonium or 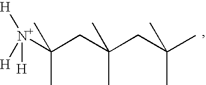
mono-, di- or tri-ethanolammonium or mono-, di- or tri-isopropanolammonium, N-methyl-N-ethanolammonium or 2-, 3- or 4-hydroxyphenylammonium.

6. A colorant according to claim 1, namely 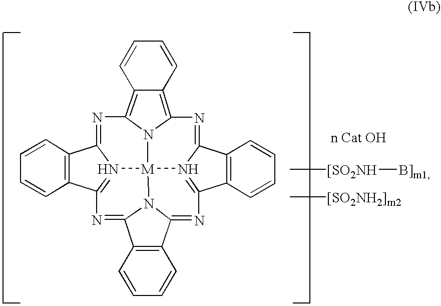
wherein M is Cu(II) or Zn(II), m1 denotes a value from 0 to 3, m2 denotes a value from 1 to 8, the sum of m1 and m2 being 3 to 5, especially 4, n denotes a value from 1 to 8, B is a group —(CH₂)ₑ-E or 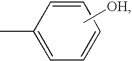
wherein e is an integer 2 or 3, E is a hydrogen atom, a group —OH, —SH, —OR¹, —SR², —NR⁵R⁶ or —C(O)OR³, and Cat is a sodium or potassium cation or unsubstituted ammonium or an ammonium cation, such as tetramethylammonium, tetraethylammonium 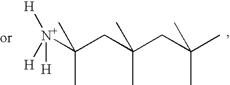
mono-, di- or tri-ethanolammonium or mono-, di- or tri-isopropanolammonium, N-methyl-N-ethanolammonium or 2-, 3- or 4-hydroxyphenylammonium, especially

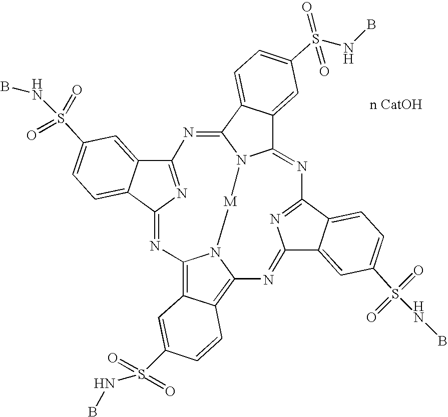

| B | M | n Cat |
|---|---|---|
| —(CH₂)₂—OH | Cu | 4 Li⁺ |
| 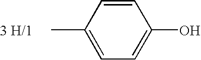 | Cu | 3 Na⁺ |
| ″ | Cu | 7 Na⁺ |
| H | Cu | 2 Na⁺ |
| —(CH₂)₂OH | Cu | 8 NH₄⁺ |
| H | Cu | 1 Na⁺ |
| 2 —(CH₂)₂OH/2 H | Cu | 1 Na⁺ |

or

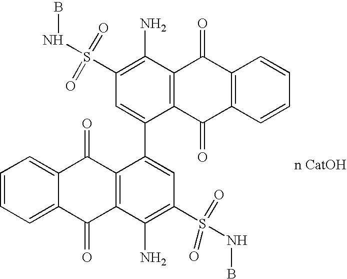

| B | n Cat |
|---|---|
| 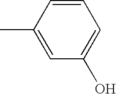 | 2 Na⁺ |
| ″ | 1 Na⁺ |
| ″ | 3 Na⁺ |
| —(CH₂)₂—OH | 1 Na⁺ |
| —(CH₂)₂—OH | 2 Na⁺ |
| H | 1 Na⁺ |
| H | 2 Na⁺ |
| 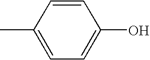 | 2 Na⁺ |

7. Use of a colorant of general formula I or II according to any one of claims 1 to 6, for the pigmenting of porous materials, especially for the pigmenting of wood or anodised aluminium in the pores, and in inkjet printing.

8. Method of pigmenting a porous material, especially for the pigmenting of wood or anodised aluminium in the pores, comprising
a) treatment of the substrate with an aqueous solution of the colorant of general formula I or II according to any one of claims 1 to 6, and
b) conversion of the colorant to a pigment of formula I′ or II′
A-[SO₂—NH—B]n+m (I′) or
A′-[NH—SO₂—B′]n′+m′ (II′),
wherein A and A′, B and B′, m and m′ and n and n′ are as defined in claim 1.

9. A porous material obtainable according to the method according to claim 8.

10. A porous material comprising pigments of general formula I′ or II′
A-[SO₂—NH—B]n+m (I′),
or
A′-[NH—SO₂—B′]n′+m′ (II′),
wherein A, B and B′, m and m′ and n and n′ are as defined in claim 1.

11. A coloured aluminium pigment that comprises platelet-shaped aluminium substrates coated with a metal oxide layer, the metal oxide layer comprising pigments of the general formula I′ or II′
A-[SO₂—NH—B]n+m (I′),
or
A′-[NH—SO₂—B′]n′+m′ (II′),
wherein A, B and B′, m and m′ and n and n′ are as defined in claim 1, and the metals of the metal layer are selected from vanadium, titanium, zirconium, silicon, aluminium and boron.
